(19) United States
(12) Patent Application Publication
(10) Pub. No.: US 2003/0109042 A1
(43) Pub. Date: Jun. 12, 2003

(54) Human ex vivo immune system

(76) Inventors: J.H. David Wu, Pittsford, NY (US); Athanassios Mantalaris, Kenton (GB)

Correspondence Address: Michael L. Goldman, Esq., NIXON PEABODY LLP, Clinton Square, P.O. Box 31051, Rochester, NY, 14603-1051, US

(21) Appl. No.: 10/244,653

(22) Filed: Sep. 16, 2002

Related U.S. Application Data
(63) Continuation of application No. 09/715,852, filed on Nov. 17, 2000, now abandoned.
(60) Provisional application No. 60/166,026, filed on Nov. 17, 1999.

(51) Int. Cl. C12N 5/08

(52) U.S. Cl. 435/372

(57) ABSTRACT

The present invention provides cultured immune system cells and methods of producing same. The method comprises culturing stromal cells and hemopoietic stem cells or in a chamber having a scaffolding covered or surrounded with culture medium, wherein the scaffolding allows for hemopoietic stem cells and stromal cells to have cell to cell contacts in three dimensions. The subject immune system cells are useful for screening drugs which inhibit or stimulate the immune system. The subject immune system cells are also useful in treating diseases of the immune system.

This application claims the benefit of U.S. Provisional Patent Application Serial No. 60/166,026, filed Nov. 17, 1999, and is a continuation of U.S. patent application Ser. No. 09/715,852, filed Nov. 17, 2000.

The invention described herein was made with the United States Government Support under National Science Foundation contract number BES-963160 and may therefore be subject to certain rights of the U.S. Government.

### FIELD OF THE INVENTION

The present invention relates to the field of cell culture and in particular, to methodologies and compositions related to cultured immune system cells.

### BACKGROUND OF THE INVENTION

All hemopoietic cell lineages, including the erythrocyte, granulocyte-macrophage, lymphocyte, and megakaryocyte, are derived from a small population of cells called pluripotent stem cells (PCSs). The PSC has the ability to self-renew or to give rise to committed stem cells that follow specific lines of hemopoietic differentiation. Specifically, the PSCs give rise to multipotential cells of the myeloid series or the lymphoid series. These multipotential cells then form the uni- or bi-potential, committed progenitor cells, which then differentiate through the precursor cells into the mature blood cells.

Stroma-mediated hemopoiesis has been demonstrated in vitro using the murine long-term bone marrow culture (LTBMC). LTBMC was first developed by Dexter and co-workers (1) employing tissue culture flasks or bottles. The mature cells produced in the Dexter LTBMC are mainly neutrophils and monocytes/macrophages. Bone marrow in vivo however, generates more than ten blood cell lineages.

Bone marrow is the hemopoietic tissue as well as a primary immune organ. A functional marrow model should therefore generate not only hemopoietic cells, but also the immune cells including lymphocytes. Presently, the only lymphopoietic model is the modified murine LTBMC developed by Whitlock and Witte (16, 17). The Whitlock-Witte culture differs from the Dexter culture in that it uses an incubation temperature of 37° C. instead of 33° C. and fetal calf serum instead of horse serum. Furthermore, the medium contains 2-mercaptoethanol but not hydrocortisone. In contrast to the Dexter culture, the Whitlock-Witte culture produces almost exclusively B-lymphocytes, indicating that the stromal cells provide a microenvironment conducive to lymphopoiesis. The Whitlock-Witte culture contains pre-B cells (producing μ heavy chains only) and mature B cells (synthesizing both light and heavy chains of IgG).

Interestingly, cultures started under the Dexter conditions, producing predominantly neutrophils and monocytes-macrophages, when switched to the Whitlock-Witte conditions, shift from myelopoiesis to lymphopoiesis (4). This shift is accompanied by regression of the fat cells and other morphological changes in the stromal layer. Therefore, stromal cells in flask culture are influenced by culture conditions to favor myelopoiesis or lymphopoiesis, but not both. The differences between the two culture systems point out the potential role of hydrocortisone in modulating lymphopoiesis. The Whitlock-Witte culture, although useful as a murine B-lymphopoiesis model, deviates from marrow in vivo in supporting the development of only one cell lineage. In addition, no human equivalent to the Whitlock-Witte culture has been reported. Although B-lymphocytes mature in bone marrow in vivo, no human bone marrow culture methods support the maturation of B-lymphocytes in vitro. Some T-cells also reside in bone marrow. Persistence of T-lymphocytes in human bone marrow culture has been reported. (14, 15). NK cells are another type of lymphoid cells generated in marrow.

Recently, a human model for in vitro B-cell lymphopoiesis has been developed (6). It is a cumbersome two-stage culture system. In the first stage, CD 34⁺ or CD34⁺ CD38- umbilical cord blood hemopoictic progenitors are cultured on the murine stromal cell line (in the presence of 2-mercaptoethanol), S17, leading to the sustained production of large numbers of CD10⁺, CD19⁺ early B-cell progenitors. In the second stage, purified CD19⁺ cells are transferred onto murine fibroblasts expressing human CD40-ligand in the presence of IL-10 and IL-4. This leads to cell proliferation and modulation of the IgM⁺ cell surface phenotype to one consistent with activated mature B cells.

The two-stage culture method presents several drawbacks. In the first instance, it requires the use of a murine stromal cell line and murine fibroblasts transfected with CD40-ligand. This creates a non-human and unnatural environment. In addition, 2-mercaptoethanol is required in the medium for the generation of early B-cell progenitors, as in the Whitlock-Witte culture. Further, it requires the presence of specific cytokines (IL-10 and IL-4) which most likely skew lymphopoiesis (6).

Presently, there is a lack of a consistent, single-stage human lymphopoiesis model which allows for the study of the intricate cell to cell interactions in lymphopoiesis, and which is not limited in the production of only B cells but which also includes other cell types present in bone marrow. The present invention provides a cell culture system for the culture of human hemopoietic stem cells and stromal cells which supports the growth and/or differentiation of the stem cells into immune system cells of all lymphocyte subtypes.

### SUMMARY OF THE INVENTION

In accordance with the present invention, it has been surprisingly discovered that all lymphocyte subtypes, including B-cells, T-cells, and NK-cells, may be produced in a three dimensional bioreactor inoculated with stromal and hemopoietic stem cells.

The present invention therefore provides a cell culture system comprising a three dimensional support for the culture of stromal and hemopoietic stem cells; and media which will support the growth and/or differentiation of the stem cells into immune system cells.

The hemopoietic stem cells may be selected from the group consisting of bone marrow stem cells, peripheral blood stem cells, embryonic stem cells, stem cells from umbilical cord and stem cells from other sources. Examples of immune system cells which may be produced in the subject cell culture system include T lymphocytes, B lymphocytes, antigen presenting cells, and natural killer cells.

Examples of T lymphocytes which may be produced in accordance with the present invention include CD4⁺ and CD8⁺ cells. The T lymphocytes produced using the cell culture system of the present invention may have αβ or γδ T cell receptors. They may be naive, activated, or memory T lymphocytes.

Examples of B lymphocytes which may be produced in the cell culture system of the present invention include CD19⁺, CD20⁺, and CD21⁺ cells. They may be IgM positive, proB, preB, IgG positive, plasma cells, and/or memory B cells. Examples of antigen presenting cells which may be produced in accordance with the present invention include macrophages and dendritic cells.

The media for use in the cell culture system of the present invention may contain cytokines or other molecules. Cytokines or other molecules which may be used in the media include for example, interleukin-2, interleukin-7, interleukin-12, slt-3L, CD40L, interleukin 4, interleukin 10, interleukin 6, BCF-1, and stem cell factor.

In accordance with the present invention, stromal and hemopoietic stem cells are used to inoculate the cell culture system. In an alternative embodiment, in addition to the stromal and hemopoietic stem cells, non-bone marrow cells may also be used to inoculate the cell culture system. Examples of non-bone marrow cells which may be used include, e.g., peripheral blood immune system cells.

The present invention provides a method of producing immune system cells which comprises culturing stromal and hemopoictic stem cells on a three dimensional support and allowing for the growth of, or differentiation into, immune system cells.

Examples of immune system cells produced by the methods of the present invention include, T lymphocytes, B lymphocytes, antigen presenting cells, natural killer cells, naive cells, activated cells, memory cells, and progenitors or precursors thereof.

Examples of T lymphocytes which may be produced by the methods of the present invention include, for example, CD4⁺, CD8⁺, CD3⁺, and TdT⁺ cells.

Examples of B lymphocytes which may be produced by the methods of the present invention include, for example, CD19⁺, CD20⁺, CD21⁺, CD10⁺, TdT⁺, CD5⁺, Ig⁺, cytoplasmic mu chain⁺ and plasma cells.

The present invention also provides a method for producing antigen specific antibodies. The method comprises culturing stromal cells and hemopoietic stem cells on a three dimensional support and allowing for the growth of, or differentiation into, immune system cells; immunizing the culture with an antigen or antigenic fragment thereof, and identifying antibodies produced by the culture system which are antigen specific. In an alternative embodiment, the culturing of stromal cells and hemopoietic stem cells may be carried out in the presence of non-bone marrow cells. In a method for producing antigen specific antibodies, the antigen or antigenic fragment thereof may be a carbohydrate, peptidoglycan, protein, glycoprotein, or a nucleic acid molecule. In a preferred embodiment, the stromal cells and hemopoictic stem cells are human cells. In accordance with the present invention, the antigen or antigenic fragment thereof may be combined with antigen presenting cells. If desired, the antigen or antigenic fragment thereof may be presented as a conjugate. Further with respect to the production of antigen specific antibodies, the immunizing of the culture may be carried out with an adjuvant.

Also with respect to producing antigen-specific antibodies, the present invention further provides methods for producing antigen specific antibodies wherein a cell line which produces a monoclonal antibody which specifically binds to the antigen is isolated.

The present invention also provides antibodies produced by the subject cultured cells. Further, the present invention provides B cells which produce the subject antibodies. Monoclonal antibodies and cell lines are also provided.

In accordance with the present invention, there is provided a method for producing antigen specific T cells. The method comprises culturing stromal cells and hemopoietic stem cells on a three dimensional support and allowing for the growth of, or differentiation into, immune system cells; immunizing the culture with an antigen or antigenic fragment thereof; and identifying T cells produced by the culture system which are antigen specific. In an alternative embodiment, culturing of stromal cells and hemopoietic stem cells may be carried out in the presence of non-bone marrow cells. Examples of antigen or antigenic fragments which may be used for immunizing the culture include a carbohydrate, peptidoglycan, protein, glycoprotein, or a nucleic acid molecule. The antigen may also be a viral antigen or a tumor antigen. The antigen or antigenic fragment thereof may be combined with antigen presenting cells and/or be presented as a conjugate. If desired, the immunizing may be carried out with an adjuvant.

The present invention also provides a method for producing dendritic cells which comprises culturing stromal cells and hemopoietic stem cells on a three dimensional support and allowing for the growth of, or differentiation into, dendritic cells. The culturing of stromal cells and hemopoietic stem cells may be carried out in the presence of non-bone marrow cells. Dendritic cells produced in accordance with the present invention may include, for example, dendritic cells from myeloid-committed precursors and dendritic cells from lymphoid-committed precursors. If desired, the culture can be selectively enriched for dendritic cells. In addition, the production of dendritic cells may be enhanced by adding one or more dendritic specific cytokines to the culture.

Examples of dendritic specific cytokines include interleukin-4, granulocyte macrophage colony stimulating factor, stem cell factor, and fins-like tyrosine kinase 3 ligand (flt-3L). Dendritic cells produced by the subject method and cell lines derived from dendritic cells are also provided.

In yet another aspect of the invention, there is provided a method for testing vaccines. The method comprises culturing stromal cells and hemopoietic stem cells on a three dimensional support and allowing for the growth of, or differentiation into, immune system cells; administering a vaccine to the cultured cells; and then determining whether the vaccine induces an immune response. If desired, the type of immune response which is induced may be determined. If desired, the culturing stromal cells and hemopoietic stem cells may be carried out in the presence of non-bone marrow cells. Also if desired, the testing for vaccines comprises screening of efficacy using cells obtained from individuals of more than one ethnic group.

In still another aspect of the invention, there is provided a method for identifying genes involved in immune system cell development and function. The method comprises altering the expression of a gene in a hemopoietic stem cell; culturing the hemopoietic stem cell and stromal cells on a three dimensional support; and determining whether the altered expression of the gene results in a phenotypic change in the cultured cells. If desired, the culturing of bone marrow cells may be carried out in the presence of non-bone marrow cells.

The present invention further provides a method for screening for genes involved in immune system cell development and function. The method comprises the steps of culturing stromal cells and hemopoietic stem cells on a three dimensional support; and identifying genes expressed in cultured cells by gene cloning techniques. If desired, the culturing of stromal cells and hemopoietic stem cells may be carried out in the presence of non-bone marrow cells. In an alternative embodiment, in addition to the steps enumerated above, the expression of the gene may be compared between cultured cells or nonimmune cells or undifferentiated cells. For example, gene expression may be compared between the cultured cells and cultured cells having a different immune cell profile.

In a related embodiment, gene expression may be compared between the cultured hemopoietic stem cells and a non-immune producing culture and genes with altered expression between the first and second cultures identified.

In yet another embodiment of the present invention, there is provided a method for determining the toxicity of a drug. The method comprises the steps of culturing stromal cells and hemopoietic stem cells on a three dimensional support and allowing for the growth of, or differentiation into, immune system cells; administering the drug to the cultured cells; and determining whether the drug is toxic to any of the cells in the culture. If desired, the culturing of stromal cells and hemopoietic stem cells may be carried out in the presence of non-bone marrow cells. The present invention also provides surviving cells resulting from the aforementioned method.

A method for determining the efficacy of a drug is also provided by the present invention. The method comprises the steps of culturing bone marrow cells on a three dimensional support and allowing for the growth of, or differentiation into immune system cells; administering the drug to the cultured cells; and determining whether the drug results in a phenotypic change in the cultured cells. If desired, the culturing of bone marrow cells may be carried out in the presence of non-bone marrow cells.

Thus in one embodiment of the method, the drug may increase the production of immune system cells. Also provided are cells which survive the method for determining the efficacy of a drug.

In another embodiment of the method, the drug may inhibit the proliferation of immune system cells.

Examples of drugs useful for performing a method of determining the efficacy of a drug include for example, nucleic acids, modified nucleic acids, antibodies, chemotherapeutic agents, and cytokines.

The present invention also provides a method for gene therapy. The method comprises the steps of culturing stromal cells and hemopoietic stem cells on a three dimensional support and allowing for the growth of, or differentiation into, immune system cells; and administering a gene to the cultured cells. If desired, the culturing of bone marrow cells may be carried out in the presence of non-bone marrow cells. In an alternative embodiment, the culture may contain helper cells which carry a vector containing the gene to be introduced. Further, the gene may be targeted to immune system cells. In a related embodiment, there are provided resultant cultured cells transformed with a gene. In a related method, the cultured cells transformed with a gene may be introduced into a patient.

In accordance with the present invention, there is also provided a method for monitoring progression of HIV infections. The method comprises culturing stromal cells and hemopoietic stem cells on a three dimensional support and allowing for the growth of, or differentiation into immune system cells; introducing HIV virus to the cultured cells; and monitoring the quantity and location of HIV in the cultured cells. If desired, the culturing of bone marrow cells may be carried out in the presence of non-bone marrow cells.

In another embodiment of the invention, there is provided a method for testing drugs which inhibit or treat HIV. The method comprises the steps of culturing stromal cells and hemopoietic stem cells on a three dimensional support and allowing for the growth of, or differentiation into, immune system cells; introducing HIV virus to the cultured cells; administering a drug to the cultured cells; and monitoring the quantity and location of HIV in the cultured cells. If desired, the culturing of stromal cells and hemopoietic stem cells may be carried out in the presence of non-bone marrow cells. The drug may be administered before, during or after the introduction of HIV to the cultured cells.

In accordance with the present invention, there is also provided a method of treating a patient which comprises the steps of administering to the patient, an effective amount of any of the immune system cells produced in the three dimensional cell culture system. Examples of such immune system cells include T lymphocytes, B lymphocytes, antigen presenting cells, natural killer cells, naive cells, activated cells, memory cells, and progenitors or precursors thereof. The aforementioned cells may be administered in any combination. If desired, only one of the aforementioned cell types may be administered.

Thus, T lymhocytes such as CD4+, CD8+, OR CD3+ cells may be administered to a patient. B lymphocytes such as CD19+, CD20+ or CD21+ cells may also be administered to a patient. Antigen presenting cells such as macrophages or dendritic cells may also be administered. B cells such as plasma cells or memory cells may also be administered to a patient.

The immune system cells produced in the three dimensional bioreactor of the present invention may be administered to a patient in an effective amount. By “effective amount” is meant an amount effective to treat the patient. As used herein, “treat” is meant to include prevent or ameliorate a condition of a patient. Thus, a patient susceptible to, or suffering from, any of the myriad of immune system conditions or disorders, may be administered the subject immune system cells or progenitors or precursors thereof, in an amount effective to prevent or ameliorate the condition or disorder. Similarly, the surviving cells obtained from the subject drug toxicity or drug efficacy assays may be administered to a patient in an effective amount.

A patient may also be treated with an antibody produced by the subject method for producing antigen specific antibodies.

The examples in this application deal with bone marrow stem cells, however it should be readily apparent that peripheral blood stem cells, embryonic stem cells, umbilical blood stem cells may, and other types of stem cells be substituted for the bone marrow stem cells.

### BRIEF DESCRIPTION OF THE DRAWINGS

FIG. 1a is a schematic drawing of one possible configuration of a three dimensional bioreactor. In the configuration pictured here, the porous or fibrous scaffolding is located in the culture chamber.

FIG. 1b is a scanning electron micrograph of a macroporous cellulose microsphere used as artificial scaffolding in the bioreactor.

FIG. 2 shows the flow cytometric analysis data of the CD10 antigen expression in the three-dimensional human bone marrow model at weeks 0 through 4.

FIG. 3 shows the flow cytometric analysis data of the CD19 and CD20 antigen expression for three-dimensional human bone marrow model at weeks 0 through 4.

FIG. 4 shows the flow cytometric analysis of the CD19 and CD21 antigen expression for the three-dimensional human bone marrow model at weeks 0 through 4.

FIG. 5A is a photomicrograph showing a TdT-positive (arrow) lymphoid progenitor cell from the 3-D reactor culture at week 1. The nucleoar TdT is stained red.

FIG. 5B is a photomicrograph showing a pre-B lymphocyte (arrow) which is stained red from cytoplasmic μ chains (the heavy chain of antibody, week 5.5).

FIG. 5C is a photomicrograph showing spots produced by the LPS-stimulated, IgG-secreting B-lymphocytes at week 4, indicating that the B-lymphocytes in the culture are functional.

FIG. 5D is the same as FIG. 5C but at a higher magnification.

FIG. 6 shows the flow cytometric analysis data of the CD3, CD4, and CD8 antigen expression for the three-dimensional human bone marrow model at weeks 0 and 4.

FIGS. 7a and 7b graphically depict cell output kinetics from the human three dimensional culture system. FIG. 7a shows the viable cell output obtained in each sampling. FIG. 7b shows the cumulative viable cell output. Cytokines used were rh IL-2 (1000U/ml), rh IL-7 (2 ng/ml), and rh SCF (50 ng/ml). No corrections have been made for depopulation of the flasks with sampling. For each culture, 6 culture chambers were inoculated. The error bars represent standard deviations.

FIG. 8 graphically depicts the results of flow cytometric analysis of the CD3, CD4, and CD8 antigen expression in the 3-D marrow culture in the absence of lymphokines. Peripheral blood mononuclear cells are denoted as PBMNC; fresh bone marrow is denoted as FBM.

FIG. 9 graphically depicts the results of flow cytometric analysis of the CD3, CD4 and CD8 antigen expression in the 3-D marrow culture in the presence of lymphokines. The cytokines added were rSCF (50 ng/ml), rh IL-2 (1000U/ml), and rh IL-7 (2 ng/ml). Peripheral blood mononuclear cells are denoted as PBMNC; fresh bone marrow is denoted as FBM.

FIG. 10 graphically depicts the results of flow cytometric analysis of the CD3, TCRαβ, and TCRγδ antigen expression in the 3-D marrow culture in the absence of lymphokines.

FIG. 11 graphically depicts the results of flow cytometric analysis of the CD3, TCRαβ, and TCRγδ antigen expression in the 3-D marrow culture in the presence of lymphokines.

FIG. 12a graphically depicts viable cell output obtained in each sampling in the 3-D marrow culture in the presence or absence of hydrocortisone. At day 10, hydrocortisone was removed from the culture medium. No corrections have been made for depopulation of the flasks with sampling. For each culture, 6 culture chambers were inoculated. The error bars represent standard deviations.

FIG. 12b graphically depicts the cumulative viable cell output. At day 10, hydrocortisone was removed from the culture medium. No corrections have been made for depopulation of the flasks with sampling. For each culture, 6 culture chambers were inoculated. The error bars represent standard deviations.

FIG. 13a is a photograph of a gel run with differential gene display products using RNA arbitrarily primed-PCR (RAP-PCR) of 4 week old adherent cells from the 3-D marrow culture in the presence (w) or absence (w/o) of hydrocortisone. The arrow identifies the location of the HRI gene fragment (682 bp). MW denotes molecular weight marker.

FIG. 13b is a photograph of the same gel in FIG. 13a with the differentially expressed genes excised.

FIG. 14 graphically depicts cell output kinetics in the 3-D marrow culture. Curves show the viable cell output obtained in each sampling. No corrections have been made for depopulation of the flasks with sampling. For each culture, 6 culture chambers were inoculated. The error bars represent standard deviations. Control cultures supplemented with medium containing animal sera are denoted as CM; cultures fed with medium containing 5% autologous plasma are denoted as 5% HP; cultures fed with medium containing 10% autologous plasma are denoted as 10% HP.

FIG. 15 shows the flow cytometric analysis data of the CD19 (immature B cells) and CD3 (T cells) antigen expression for the three-dimensional human bone marrow model. Cultures fed with medium supplemented with animal sera denoted as CM; cultures fed with medium supplemented with 10% human plasma denoted as 10% HP.

FIG. 16 graphically depict flow cytometric analysis of the CD19 (immature B cells) and CD3 (T cells) antigen expression for the three dimensional human bone marrow model. Peripheral blood mononuclear cells are denoted as PBMNC, cultures fed with medium supplemented with animal sera denoted as CM; cultures fed with culture medium supplemented with 5% autologous plasma denoted as 5% HP; cultures fed with culture medium supplemented with 10% autologous plasma denoted as 10% HP.

FIG. 17a shows the differential cell output kinetics of the nonadherent erythroid cells recovered from the human three-dimensional bone marrow culture. The differential cell analysis was performed blindly by counting over 100 cells per sample. Cultures fed with medium supplemented with animal sera are denoted as CM; cultures fed with medium supplemented with 10% autologous plasma are denoted as 10% HP.

FIG. 17b shows the differential cell output kinetics of the nonadherent myeloid cells recovered from the human three-dimensional bone marrow culture. Analysis performed and notations are as described for FIG. 17a.

FIG. 17c shows the differential cell output kinetics of the nonadherent lymphoid cells recovered from the human three-dimensional bone marrow culture. Analysis performed and notations are as described for FIG. 17a.

### DETAILED DESCRIPTION OF THE INVENTION

In accordance with the present invention, it has been surprisingly discovered that all lymphocyte subtypes including B-cells, T-cells, and NK-cells, may be produced in a three dimensional bioreactor inoculated with stromal and hemopoietic stem cells.

The present invention therefore provides a cell culture system comprising a three dimensional support for the culture of hemopoietic stem cells and stromal cells, and media which supports the growth of, or differentiation of, the stem cells into immune system cells. As used herein, “immune system cells” is meant to include T lymphocytes (T-cells), B lymphocytes (B-cells), antigen presenting cells, and natural killer cells (NK-cells).

The culture system comprises a chamber or container having a scaffolding covered or surrounded in culture medium wherein the scaffolding allows for the hemopoietic stem cells and stromal cells to have cell to cell contacts in three dimensions.

As used herein, the term “hemopoictic stem cells” include bone marrow stem cells, peripheral stem cells, embryonic stem cells, umbilical blood stem cells and other types of stem cells. As used herein, “stromal cells” may include such cells as endothelial cells, reticular cells, fat cells and professional antigen presenting cells such as dendritic cells. The stromal cells may be isolated from many different sources such as e.g., adult and fetal bone marrow, spleen, thymus, peripheral blood, liver, umbilical cord, para-aortic splanchnopleura, aorta, gonads and mesonephros (AGM), lymph node, and other types of stromal cells, or derived from stem cells such as e.g., bone marrow stem cells, peripheral blood cells, peripheral stem cells, embryonic stem cells, umbilical cord cells, umbilical blood stem cells, embryonic stem cells, other types of stem cells, or any combination of these cells.

In accordance with the present invention, a bioreactor system and method for generating immune system cells is provided. The bioreactor of the present invention provides a three-dimensional structure which mimics the natural extracellular matrix and ample surface area of the bone marrow and allows cell to cell interaction at a tissue-like cell density. It is understood that the bioreactor of the present invention may have many different configurations so long as it provides a three-dimensional structure. With respect to the bioreactor, the term “three-dimensional structure” is used interchangeably with the term “scaffolding”.

The bioreactor for use in generating immune system cells comprises a container or vessel having at least one chamber or section with scaffolding located therein. The scaffolding is made of a porous or fibrous substrate. Culture media is placed over or around the porous or fibrous substrate.

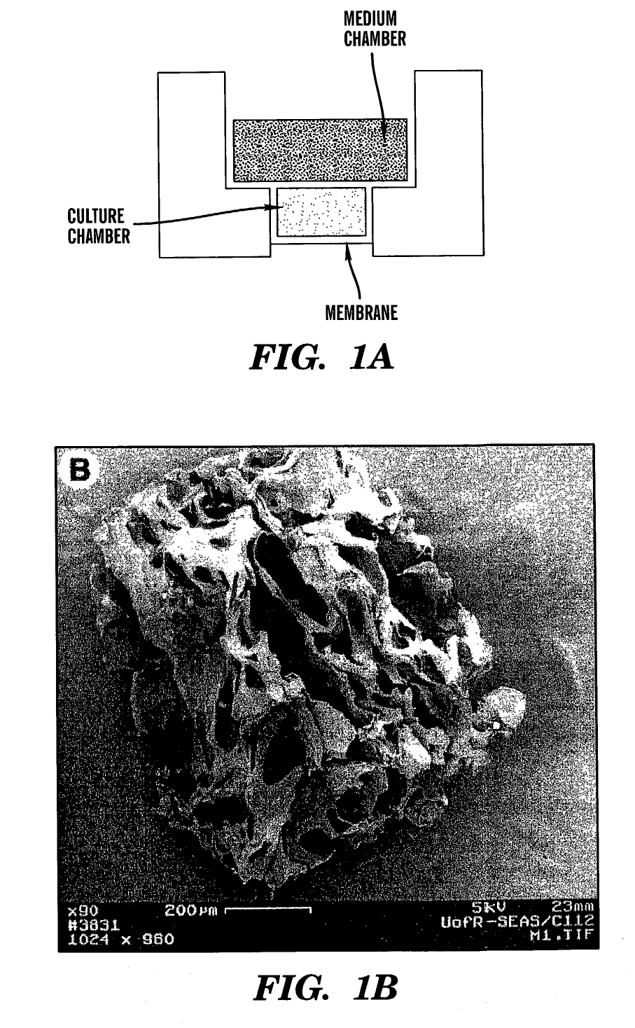

FIG. 1a illustrates one possible configuration of a bioreactor which may be used to generate immune system cells. In FIG. 1, the porous or fibrous scaffolding is located in a lower, culture chamber. It is understood that the bioreactor of the present invention may have any number of configurations so long as it provides a three dimensional structure (scaffolding).

The walls of the container or vessel may comprise any number of materials such as glass, ceramic, plastic, polycarbonate, vinyl, polyvinyl chloride (PVC), metal, etc. Culture medium which will support the growth immune system cells and/or the differentiation of hemopoietic stem cells and stromal cells into immune system cells is placed over and/or around the porous or fibrous material.

Many different porous or fibrous materials may be used as scaffolding in the bioreactor such as, e.g., tangled fibers, porous particles, sponge, or sponge-like material. The porous or fibrous scaffolding allows hemopoietic stem cells and/stromal cells to lodge onto, proliferate and differentiate. For purposes of example only and not limitation, suitable scaffolding substrates may be prepared using a wide variety of materials including natural polymers such as polysaccharides and fibrous proteins, synthetic polymers such as polyamides (nylon), polyesters, polyurethanes, degradable polymers such as PGA, PGLA, and minerals including ceramics and metals, coral, gelatin, polyacrylamide, cotton, glass fiber, corrageenans, alginate, chitin, and dextrans. Examples of tangled fibers include glass wool, steel wool, and wire or fibrous mesh.

Examples of porous particles include, e.g., beads, slabs, cubes, and cylinders (made from glass, plastic, or the like) cellulose, agar, hydroxyapatite, treated or untreated bone, collagen, gels such as Sephacryl, Sephadex, Sepharose, agarose or polyacrylamide. “Treated” bone may be subjected to different chemicals such as e.g., acid or alkali solutions. Such treatment alters the porosity of bone. If desired, the substrate may be coated with an extracellular matrix or matrices, such as, e.g., collagen, matrigel, fibronectin, heparin sulfate, hyalumonic and chondroitin sulfate, laminin, hemonectin, or proteoglycans.

The fibrous or porous material used as scaffolding in the bioreactor forms openings or pores into which hemopoietic stem cells and stromal cells enter. Once entered, the cells become entrapped or adhered to the fibrous or porous material and colonize and/or aggregate thereon. Cell attachment and colonization can occur merely by inoculating the cells into the culture medium which overlays and/or surrounds the porous or fibrous substrate. Cell attachment and colonization may also occur by inoculating the cells directly onto the porous or fibrous substrates.

In accordance with the present invention, hemopoietic stem cells and stromal cells must be able to enter the openings (pores) of the fibrous or porous material. The skilled artisan is cognizant of the different sizes of hemopoietic stem cells and stromal cells and therefore the pore size needed to accommodate such cells. Generally speaking, a pore size in the range of from about 15 microns to about 1000 microns may be used. Preferably, a pore size in the range of from about 100 microns to about 300 microns is used.

In a preferred embodiment, a membrane is placed in the bioreactor in order to facilitate gas exchange. The membrane is gas permeable and may have a thickness in the range of from about 10 to about 100 μm. In a more preferred embodiment, the membrane has a thickness of about 50 μm. The membrane is placed over an opening in the bottom or side of the chamber or container. In order to prevent excessive leakage of media and cells from the bioreactor, a gasket may be placed around the opening and/or a solid plate placed under or alongside the opening and the assembly fastened.

The cell medium used in the bioreactor may be any of the widely known media used to support growth and/or differentiation of bone marrow cells, and in particular, growth and differentiation of hemopoietic stem cells and stromal cells into immune system cells. For example, the following classical media may be used and supplemented, if desired, with vitamin and amino acid solutions, serum, and/or antibiotics: Fisher's medium (Gibco), Basal Media Eagle (BME), Dulbecco's Modified Eagle Media (D-MEM), Iscoves's Modified Dulbecco's Media, Minimum Essential Media (MEM), McCoy's 5A Media, and RPMI Media.

Specialized media may also be used such as e.g., MyeloCult™ (Stem Cell Technologies), and Opti-Cell™ (ICN Biomedicals). If desired, serum free media may be used such as, e.g., StemSpan SFEM™ (StemCell Technologies), StemPro 34 SFM (Life Technologies) and Marrow-Gro (Quality Biological Inc.).

In a preferred embodiment, McCoy's 5A medium (Gibco) is used at about 70% v/v, supplemented with vitamin and amino acid solutions. In an even more preferred embodiment, the culture medium comprises approximately 70% (v/v) McCoy's 5A medium (Gibco), approximately 1×10⁻⁶ M hydrocortisone, approximately 50 ug/ml penicillin, approximately 50 mg/ml streptomycin, approximately 0.2 mM L-glutamine, approximately 0.45% sodium bicarbonate, approximately 1×MEM sodium pyruvate, approximately 1×MEM vitamin solution, approximately 0.4×MEM amino acid solution, approximately 12.5% (v/v) heat inactivated horse serum and approximately 12.5% heat inactivated FBS. The medium chamber may be continuously perfused if desired. The dissolved oxygen concentration and pH of the media may be controlled by well known methods.

The bioreactor is inoculated with hemopoictic stem cells and stromal cells by gently adding e.g., pipetting, into the three-dimensional scaffolding portion of the bioreactor. Alternatively, the hemopoietic stem cells and stromal cells may be added to the culture covering and/or surrounding the three dimensional scaffolding. Cells will settle or migrate into the porous or fibrous material making up the scaffolding. The number of cells added to the bioreactor depends on the total area of the three-dimensional scaffolding and volume of culture media. Preferably, hemopoietic stem cells and stromal cells isolated from any of the sources discussed extensively herein, are centrifuged through a gradient such as a Ficol/Paque to remove mature red blood cells, yielding mononuclear cells.

For a bioreactor having a culture chamber of about 3/16″ height by about 5/16″ width by about 5/16″ length and packed with about 0.01 g of a porous or fibrous substrate, the number of mononuclear cells added to the bioreactor may be anywhere in the range of from about 10⁴ to 10⁹mononuclear cells. Preferably, 4-6×10⁶ cells may be used to inoculate the bioreactor. Using these guidelines, one skilled in the art is able to adjust the number of cells used to inoculate the bioreactor depending on the total area of the three-dimensional scaffolding, volume of culture media, type of three-dimensional scaffolding, and source of hemopoietic and stromal cells.

The culture may be fed every second day with the culture medium. Various other ingredients may be added to the culture media. Such media is herein termed “supplemented”. The media may contain cytokines, extracellular matrices, or other biologically active molecules. Thus for example, recombinant stem cell factor (rSCF), and the lymphocyte-specific lymphokines, interleukin 2 (rh IL-2) and interleukin 7, may be added to the culture media. For example, rSCF may be added in the approximate amount of about 50 ng/ml. Interleukin 2 may be added in an approximate amount of about 1000 U per ml. Interleukin 7 may be added in an approximate amount of about 2 ng/ml. The aforementioned amounts are exemplary and empirical. The skilled artisan may therefore vary the amounts according to the bioreactor setup i.e., size, volume, number and source of cells. In a preferred embodiment, the cultures are fed daily with unsupplemented medium and every second day with the supplemented medium.

The cell culture is allowed to grow anywhere from about a few days to several weeks. Preferably, the cultures are harvested after about one week to about four or five weeks. Hydrocortisone is also preferably removed from the culture medium anywhere from about one to three weeks to avoid potential inhibition of immune system cell differentiation. In an alternative embodiment, hydrocortisone is not added to the media at all.

The present invention thus provides a method of producing immune system cells which comprises culturing stromal and hemopoietic stem cells on a three dimensional support and allowing for the growth of, or differentiation into, immune system cells.

Examples of immune system cells produced by the methods of the present invention include, T lymphocytes, B lymphocytes, antigen presenting cells, natural killer cells, naive cells, activated cells, memory cells, and progenitors or precursors thereof.

Examples of T lymphocytes which may be produced by the methods of the present invention include, for example, CD4⁺, CD8⁺, CD3⁺, and TdT⁺ cells.

Examples of B lymphocytes which may be produced by the methods of the present invention include, for example, CD19⁺, CD20⁺, CD21⁺, CD10⁺, TdT⁺, CD5⁺, Ig⁺, cytoplasmic mu chain⁺ and plasma cells.

Immune cells may be harvested in any number of well known methods. The chamber may be treated with any suitable agent, such as collagenase, to release the adhering cells. Non-adhering cells may be collected as they release into the medium. Cells may also be removed from the substrate by physical means such as shaking, agitation, etc. Thereafter, the cells are collected using any known procedure in the art such as e.g., pipetting or centrifugation. Preferably, non-adherent cells are released by gentle stirring and mixing the bed of porous or fibrous material and then collected by centrifugation or sedimentation.

If desired, the cell samples collected from the bioreactor may be further enriched for immune system cells using well known methods of positive selection. Thus, for example, a solid support (such as beads) having an antibody that binds immune system cells conjugated thereto, may be mixed with the cell sample. In this way the three immune system cell types may be isolated together or separately. If a mixed population of lymphocytes is desired, then the solid support should be conjugating to antibodies for all subtypes. If a particular subtype is desired, then a solid support having an antibody conjugated thereto which binds a particular lymphocyte may be used. Examples of antibodies which may be conjugated to a solid support include anti-CD3⁺, anti-CD4⁺ (for helper T-cells), anti-CD8⁺ (for cytotoxic T-cells), anti-CD19⁺ (for immature B-cells), anti-CD19⁺, anti-CD20⁺ (for mature B-cells) anti-TdT anticytoplasmic, anti-surface IgG and anti-surface IgM (for antigen stimulated B-cells). Antibody conjugated beads with immune system cells bound thereto are then collected by gravity or other means such as a magnet, in the case of magnetic beads.

Negative selection may also be used as a means of enriching the immune system cell population and subpopulations, e.g., B-cells, T-cells, and NK-cells in the cell sample removed from the bioreactor. With a negative selection scheme, a solid support (such as beads) having conjugated thereto one or more antibodies which react with cells other than immune system cells, may be mixed with the cell sample. Antibody conjugated beads with cells other than immune system cells bound thereto are then collected by gravity or other means such as a magnet, in the case of magnetic beads.

Immune system cells may be identified using any well known method such as e.g., flow-cytometry analysis, immunocytochemistry, enzyme-linked immunospot (ELISPOT), and cytotoxicity assay for NK cells. These methodologies are well known in the art and described herein.

The cultured immune system cells of the present invention have a myriad of uses in the therapeutic, diagnostic, and clinical settings. For example, the subject immune system cells may be used to produce antigen specific antibodies. Thus in accordance with the present invention, there is provided a method for producing antigen specific antibodies. The method comprises culturing hemopoietic stem cells and stromal cells on a three dimensional support for a time and under conditions sufficient for the growth of, and/or differentiation into immune cells; immunizing the culture with an antigen or antigenic fragment thereof, and identifying antibodies produced by the culture system which are antigen-specific. The antigen or antigenic fragment can include, for example, a carbohydrate, peptidoglycan, protein, glycoprotein, virus, tissue mass, cell, cell fragment, or a nucleic acid molecule. The virus, tissue mass, cell, or cell fragment may be live or dead. Any substance which can induce antibody production may be used. Example 3 describes the production of antibodies in the culture system of the present invention by immunizing with a lipopolysaccharide (LPS).

Methods of immunizing cells are well known in the art and are described for example, in Fundamental Immunology 1993, Raven Press, New York, W. E. Paul, ed., which is incorporated by reference herein as if fully set forth. Methods of identifying antibodies which are antigen specific are well known and include, for example, ELISA, ELISPOT, and PCR.

The hemopoietic stem cells may be for example, as previously described, bone marrow stem cells. However, other cells such as peripheral blood stem cells, embryonic stem cells, stem cells from umbilical cord, and stem cells from other sources may also be used. Preferably, the hemopoietic stem cells are human cells. If desired, the antigen or antigenic fragment thereof may be combined with antigen presenting cells. In addition, the antigen or antigenic fragment may be presented as a conjugate. Examples of conjugates include diphtheria and tetanus oxoids. Immunization may be carried out with an adjuvant if desired. An example of an adjuvant which may be used in the present invention includes Freund's.

Also in accordance with the present invention, there are provided antibodies produced by the methods described hereinabove. Monoclonal antibodies are usually produced using well known methods such as those originally described by Milstein and Kohler (1975) Nature 256:495-497. In the prior art procedures, a mouse or suitable animal is injected with an antigen or fragment thereof. The animal is subsequently sacrificed and spleen cells are fused with myeloma cells to produce a hybridoma. In accordance with the present invention, the antibody producing B-cells removed from the bioreactor may be screened to isolate individual cells which secrete a singly antibody species to the antigen. Cell lines may then be derived which secrete the monoclonal antibody.

B cells and B cell lines which produce the subject antibodies may be isolated using well known methods such as those described in Fundamental Immunology 1993, Raven Press, New York, W. E. Paul, ed.

The present invention also provides a method for producing antigen specific T cells. The method comprises the steps of culturing stromal cells and hemopoictic stem cells on a three dimensional support and allowing for growth of, or differentiation into, immune system cells; immunizing the culture with an antigen or antigenic fragment thereof, and identifying T cells produced by the culture which are antigen specific. T cells may be identified using well known methods in the art such as immunocytochemistry for T cell receptors. For example, using immunocytochemistry for CD4+, CD8+, αβ, or γδ, T cells may be identified.

Thus for example, an antigen or antigenic fragment used to immunize the culture in a method for producing antigen specific T cells, may be a carbohydrate, peptidoglycan, protein, glycoprotein, virus, tissue mass, cell, cell fragment, or a nucleic acid molecule. The virus, tissue mass, cell, or cell fragment may be live or dead. The antigen may also be a viral antigen or a tumor antigen.

The hemopoietic stem cells may be for example, as previously described, bone marrow cells. However, other cells such as peripheral blood stem cells, embryonic stem cells, stem cells from umbilical cord or stem cells from other sources may also be used. Preferably, the hemopoietic stem cells are human cells. If desired, the antigen or antigenic fragment thereof may be combined with antigen presenting cells. In addition, the antigen or antigenic fragment may be presented as a conjugate. Examples of conjugates include diphtheria and tetanus oxoids. Immunization may be carried out with an adjuvant such as Freund's.

In accordance with the present invention, there is also provided a method for producing dendritic cells. The method comprises culturing stromal cells and hemopoietic stem cells on a three dimensional support and allowing for the growth of, and/or differentiation into, dendritic cells. As described hereinbefore, the hemopoietic stem cells may be for example, bone marrow cells. However, other cells such as peripheral blood stem cells, embryonic stem cells, stem cells from umbilical cord and stem cells from other sources may also be used. Preferably, the hemopoietic stem cells are human cells. If desired, the culturing of hemopoietic stem cells may be carried out in the presence of non-bone marrow cells.

Examples of dendritic cells which may be produced in accordance with the present invention include for example, dendritic cells from myeloid-committed precursors and dendritic cells from lymphoid-committed precursors.

If desired, after culturing the stromal cells and hemopoietic stem cells on the three dimensional support and allowing for the growth of, and/or differentiation into dendritic cells, the dendritic cell population may be selectively enriched. Selective enhancement of dendritic cells may be performed by addition of a dendritic specific cytokine to the culture. Examples of dendritic specific cytokines include, interleukin-4, macrophage colony stimulating factor, stem cell factor, and fms-like tyrosine kinase 3 ligand.

The present invention therefore also provides dendritic cells produced by the method of culturing stromal cells and hemopoietic stem cells on a three dimensional support and allowing for the growth of, and/or differentiation into, dendritic cells. Likewise, the present invention provides a dendritic cell line produced by a method of culturing hemopoietic stem cells on a three dimensional support, allowing for the growth of, and/or differentiation into, dendritic cells and enhancing the production of a dendritic cell line by the addition of dendritic specific cytokine to the culture. Dendritic cells produced in accordance with the present invention may be isolated for example, by negative selection using immunomagnetic isolation methods.

Also in accordance with the present invention, there is provided a method for testing vaccines. The method comprises the steps of culturing stromal cells and hemopoietic stem cells on a three dimensional support and allowing for the growth of, and/or differentiation into immune system cells, administering a vaccine to the cultured cells, and determining whether the vaccine induces an immune response. If desired, the culturing of hemopoietic cells may be carried out in the presence of non-bone marrow cells. As used herein, “vaccine” is meant to include any substance that induces an immune response, i.e., the activation of immune system cells. The type of immune response induced by the vaccine may be determined using well known methods such as ELISA and flow cytometry. In an alternative embodiment, the method of testing a vaccine by method herein described may further comprise screening of efficacy using cells obtained from individuals of more than one ethnic group. For example, the screening may comprise cytotoxicity assays.

The present invention also provides a method for identifying genes involved in immune system cell development and function. The method comprises altering the expression of a gene in a hemopoictic stem cell, culturing the cell on a three dimensional support, and determining whether the altered expression of the gene results in a phenotypic change in the cultured cells. If desired, the method may be carried out in the presence of non-bone marrow cells. Examples of phenotypic changes which may be detected include for example, changes in surface marker expression and cytokine/chemokine expression. Such changes in phenotype may be detected using techniques such as flow cytometry, immunocytochemistry, ELISPOT assay for antibody production cells.

In yet another aspect of the invention, there is provided a method of screening for genes involved in immune system cell development and function. In accordance with this method, the expression of a gene in a hemopoietic stem cell is altered and the hemopoietic stem cell(s) and stromal cells cultured on a three dimensional support. A determination is then made as to whether the altered expression of the gene results in a phenotypic change in the cultured cells.

Expression of a gene in a hemopoietic stem cell may be altered by any of the well known methods. For example, a hemopoietic stem cell may be transformed with a genetic construct comprising a sequence which inserts itself into a gene. If the gene into which the sequence inserts itself is a gene involved in immune system cell development and function, the insertion of the foreign genetic sequence interrupts the gene and may manifest itself by a phenotypic change. Alternatively, an antisense molecule may be used to target a gene involved in immune system cell development and function. If transformation of a hemopoietic stem cell with an antisense molecule results in a phenotypic change in the hemopoietic stem cell, then it may be deduced that the molecule targets a gene involved in immune system cell development and function. Naked DNA or RNA may also be used to transfect bone marrow cells. Cells may be transfected for example, by retroviruses.

There are many different methods of altering the expression of a gene in a hemopoietic stem cell. Besides the gene interruption and antisense strategies described hereinabove, mutagenesis may also be used. Thus for example, hemopoietic stem cells may be contacted or exposed to a mutagen, grown in the three dimensional support, and then a determination made as to whether the mutagenized cells result in a phenotypic change in the cultured cells.

If desired, the culturing of stromal cells and hemopoietic stem cells may be carried out in the presence of non-bone marrow cells. In an alternative embodiment, the expression of the gene in the cultured cells may be compared to non-immune system cells or undifferentiated cells. Such a comparison has the purpose of examining their cellular function in relation to the gene of interest. In yet another embodiment, after comparing the expression of the gene in the cultured cells to genes of cells in a non-immune producing culture, genes with altered expression between the first and second cultures are identified. In still another embodiment, the expression of the gene in cultured cells may be compared to cells having a different immune cell profile.

In accordance with the present invention, there are provided methods for determining the toxicity or efficacy of a drug. In this aspect of the invention, stromal cells and hemopoietic stem cells are cultured on a three dimensional support and allowed to differentiate into immune system cells. A drug is administered to the cultured cells, and a determination is then made as to whether the drug is toxic to any of the cells in the culture. If the drug is either non-toxic or marginally toxic, a determination as to efficacy can then be made. As used herein, “drug” encompasses any element, molecule, chemical compound, hormone, growth factor, nucleotide sequence (including oligonucleotides), protein (including peptides), or reagents which have the ability to affect immune system cells. Thus for example, B cells may be affected in their ability to produce antibodies. T cells may be affected in their ability to mediate their cellular immunity functions, such as cytotoxicity. NK cells may be affected in their lytic activity. The present invention thus also provides immune system cells which have been exposed to a drug and which have survived such exposure.

In a typical toxicity or efficacy assay for a drug which affects immune system cells, cultured immune system cells are removed from the bioreactor and placed in a petri dish, flask, microscope slide, microtiter dish or the like with enough culture medium or buffered solution to keep the cells alive. Cultured immune system cells may comprise mixed populations of cells, e.g., T cells, B cells, NK cells, and the like. Alternatively, subpopulations may be isolated and used in the toxicity assays. Preferably, a pH of approximately 7.2, and a temperature of about 37° C. is maintained. The number of immune system cells which may be used in a screening assay is empirical. Typically, a sample containing 1×10⁶ total cells may be used, depending upon the number of immune system cells in the cell sample.

The number of immune system cells in a cell sample relative to other cells may be determined microscopically by counting cells or immunohistochemically as described. herein. Methods of cell counting are well known in the art and are also described in Example 1, “Differential Cell Counts”. The concentration of the drug to be tested for toxicity or efficacy is empirical. One skilled in the art is familiar with methods of adjusting concentrations of different compositions in order to best identify the effects of a test compound in the screening assay. Typically, a range of concentrations is used and those portions of the range which exhibit serious deleterious effects on immune system cell viability eliminated for further study. Those portions of the range having less deleterious effects on immune system cell viability are identified and used to further determine efficacy.

The mixture of immune system cells and drug is incubated for a time and under conditions sufficient for the inhibition or stimulation of immune function to be carried out. As defined herein, a sufficient time can be anywhere from about five minutes to several hours or more. When immune system cells are tested in a petri dish, flask, microscope slide, microtiter dish or the like, a sufficient time may be several minutes to several hours. Of course, the test time may be extended if needed in order to see effects on the cells. The skilled artisan is able to determine the optimal time for running the screening assay by removing samples and examining cells microscopically for viability.

A preferred buffer for use in the reactions is Phenol red-free MEM supplemented with 1×nonessential amino acids, 1×L-glutamine, 10% FBS, 50U/ml penicillin and 50 μg/ml streptomycin. In a preferred embodiment, the test reaction volume is between about 0.5 and about 2 ml. In a more preferred embodiment, the reaction volume is about 1 ml. In a preferred embodiment, the incubation temperature is approximately 37° C.

The test compound may be added to the culture medium or into the three dimensional scaffolding. The time at which the test compound is added is empirical but is relatively early. Typically, control runs are performed in which no test compounds are added to the bioreactor.

Examples of drugs which may tested for toxicity and efficacy by the methods of the present invention include for example, nucleic acids, modified nucleic acids, antibodies, chemotherapeutic agents, and cytokines. As described above, however, any available test compound may be used to screen for toxicity and/or efficacy on immune system cells. In some cases, the classification of a test compound as potential inhibitor or potential stimulator (inducer) of immune system cells is unknown and is initially determined by the assay.

The present invention also provides a method for gene therapy. The method comprises culturing stromal and hemopoietic stem cells on a three dimensional support, allowing for the growth of, and/or differentiation into immune system cells, and then administering a gene to the cultured cells. If desired, the culturing of stromal and hemopoietic stem cells may be carried out in the presence of non-bone marrow cells. By “administering” a gene to cultured cells, it is meant that the gene is used to transfect a cultured cell. In this aspect of the invention, the gene therapy may be thought of as ex vivo gene therapy. Methods of transfecting mammalian cells, including bone marrow cells are known in the art. See e.g., “Retrovirus transformed hemopoietic progenitors” in Immunology Methods Manual, 1997 Academic Press, San Diego, I. Lefkovits, ed. Transformed hemopoictic stem cells made in accordance with the method described herein are also provided. In an alternative embodiment, the culture contains helper cells which carry a vector containing the gene to be introduced.

The present invention also provides a method wherein the transfected hemopoietic stem cells are introduced into a patient. Introduction may be by any number of methods such as transplantation to a particular cite in the body, such as a particular tissue or organ. In a preferred embodiment, the site is the bone marrow. Systemic infusion of cells may also be performed.

In another embodiment, the gene may be targeted to immune system cells. Methods of targeting to immune system cells include the used of retroviruses.

The present invention also provides a method for monitoring progression of HIV infections. In this aspect of the invention, the method comprises the steps of culturing stromal cells and hemopoietic stem cells on a three dimensional support and allowing for the growth of, and/or differentiation into immune system cells, introducing HIV virus to the cultured cells, and monitoring the quantity and location of HIV in the cultured cells. Again, if desired, the culturing of stomal cells and hemopoietic stem cells may be carried out in the presence of non-bone marrow cells.

Also provided by the present invention is a method for testing drugs which inhibit or treat HIV. The method comprises culturing stromal and hemopoietic stem cells on a three dimensional support and allowing for the growth of, and/or differentiation into immune system cells; introducing HIV virus to the cultured cells, administering a drug to the cultured cells, and monitoring the quantity and location of HIV in the cultured cells. The drug may be administered before or after introducing HIV to the cultured cells. Again, if desired, the culturing of bone marrow cells may be carried out in the presence of non-bone marrow cells.

In accordance with the present invention, there is also provided a method of treating a patient which comprises the steps of administering to the patient, an effective amount of any of the immune system cells produced in the three dimensional cell culture system. Examples of such immune system cells include T lymphocytes, B lymphocytes, antigen presenting cells, natural killer cells, naive cells, activated cells, memory cells, and progenitors or precursors thereof. The aforementioned cells may be administered in any combination. If desired, only one of the aforementioned cell types may be administered.

Thus, T lymphocytes such as CD4+, CD8+, CD3+ or TdT cells may be administered to a patient. B lymphocytes such as CD19+, CD20+ or CD21+ cells may also be administered to a patient. Antigen presenting cells such as macrophages or dendritic cells may also be administered. B cells such as plasma cells or memory cells may also be administered to a patient.

The immune system cells produced in the three dimensional bioreactor of the present invention may be administered to a patient in an effective amount. By “effective amount” is meant an amount effective to treat the patient. As used herein, “treat” is meant to include prevent or ameliorate a condition of a patient. Thus, a patient susceptible to, or suffering from, any of the myriad of immune system conditions or disorders, may be administered the subject immune system cells or progenitors or precursors thereof, in an amount effective to prevent or ameliorate the condition or disorder. Examples of immune system conditions and disorders include, for example, acquired immune deficiency syndrome (AIDS), hemophilia, and DiGeorge's syndrome.

Similarly, the surviving cells obtained from the subject drug toxicity or drug efficacy assays may be administered to a patient in an effective amount.

A patient may also be treated with an effective amount of an antibody produced by the subject method for producing antigen specific antibodies. By “effective amount” is meant an amount effective to neutralize the contaminating (foreign) antigen.

The present invention also provides a method of immune cell maturation, selection, antigen presentation, or expansion. The method comprises removing the immune cells produced in the three dimensional bioreactor and inoculating a further culture with the removed immune cells. Matured expanded, and/or antigen-presenting cells may be removed and selected from the further cell culture using well known methods as well as methods described herein. As used herein, “further cell culture” may include a three dimensional support (scaffolding), media which will support the growth of, or differentiation of hemopoietic stem cells into immune system cells; i.e., a second three dimensional bioreactor.

Preferably, “further cell culture” is meant to include at least one of an adult or fetal spleen cell culture, a thymus cell culture, a lymph node cell culture, or liver cell culture system. Methods of culturing adult or fetal spleen cells, thymus cells, lymph node cells or liver cells are well known in the art.

The present invention also provides a method of B cell maturation, selection, antigen-presentation or expansion which comprises inoculating a further culture with antibody producing B cells produced in the subject three dimensional bioreactor. The antibody producing B cells are produced by culturing stromal and hemopoietic stem cells on a three dimensional support, allowing for the growth of, or differentiation into immune system cells, immunizing the culture with an antigen or antigenic fragment thereof, and identifying the antibodies produced and isolating the B cells producing the antigen specific antibodies.

In yet another aspect of the invention, there is provided a method of T cell maturation, selection, antigen-presentation. The method comprises inoculating a further cell culture with antigen specific T cells. The antigen specific T cells are produced by culturing stromal and hemopoietic stem cells on a three dimensional support, allowing for the growth of, or differentiation into immune system cells, immunizing the culture with an antigen or antigenic fragment thereof, and identifying the antibodies produced and isolating the T cells produced by the culture which are antigen specific.

The present invention provides a method of dendritic cell maturation, selection, antigen-charging, or expansion. The method comprises removing immune system cells from the three dimensional bioreactor, isolating dendritic cells, and inoculating a further cell culture with the dendritic cells.

The present invention further provides a method of natural killer cell maturation, selection, antigen presentation or expansion. The method comprises removing immune system cells from the three dimensional bioreactor, isolating natural killer cells, and inoculating a further cell culture with the natural killer cells.

Also in accordance with the present invention, there is provided a method of treating a patient which comprises administering an effective amount of the natural killer cells from the further cell culture.

In yet another aspect of the invention, there is provided a method of cell growth and expansion which comprises culturing stromal and hemopoietic stem cells on a three dimensional support and allowing for the growth of, or differentiation into, immune system cells. The immune system cells are then transfected with a nucleic acid sequence and the transfected cells used to inoculate a further cell culture.

In yet another aspect of the invention, there is provided a method for HIV-infected cell growth and expansion which comprises culturing stromal and hemopoietic stem cells on a three dimensional support and allowing for the growth of, or differentiation into, immune system cells. HIV is then introduced into the cultured cells and the HIV infected cells are used to inoculate a further cell culture.

In still another aspect of the invention, there is provided a method of cell growth and expansion which comprises culturing stromal and hemopoietic stem cells on a three dimensional support and allowing for the growth of, or differentiation into, immune cells. HIV is then introduced into the cultured cells and a drug is also introduced into the cultured cells. The HIV-infected and drug exposed cells are then used to inoculate a further cell culture.

The invention is further illustrated by the following specific examples which are not intended in any way to limit the scope of the invention.

### General Materials and Methods

Flow-Cytometry Analysis

Percentages of lymphocyte subtypes (helper and cytolytic T cells and B cells) and activation lymphocyte surface markers were quantified by flow-cytometry on an EPICS Profile Analyzer (Coulter, Miami, Fla.). Cell samples were incubated with fluorescence-labeled antibodies and isotype controls. Antibodies used were anti-CD3 (pan T cell), anti-CD4 (helper T cell), anti-CD8 (cytolytic T cell), anti-TCRαβ (T-cells with αβ T cell receptor), anti-TCRγδ (T cells with γδ T cell receptor), anti-CD45RA (naïve T cells), anti-CD45RO (activated T cells), anti-CD19, anti-CD20, anti-CD21, and anti-CD10 (B cells) (10).

Immunocytochemistry

Acetone-fixed cytospin slide preparations of the nonadherent cells from the cultures were labeled with monoclonal antibodies (anti-CD3, anti-CD19, anti-CD56, and anti-TdT) or polyclonal antibodies (anti-cytoplasmic μ, anti-surface IgG, and anti-surface IgM), followed by a biotin-conjugated secondary antibody and streptavidin-conjugated peroxidase (DPC). Endogenous peroxidase activities were quenched by immersing the slides in 3% hydrogen peroxide for 5 minutes prior to the immunostaining (8). Positively stained cells were identified under a light microscope. The morphological characteristics of the positively stained cells were also examined to ensure a consistency with their respective subtypes defined by the cytochemistry.

ELISPOT Assay for Antibody-Producing B-Cells

By using the ELISPOT (enzyme-linked immunospot) assay (10), the immunoglobulin-producing B-cells were detected. Briefly, antigen was coated to a solid phase (petri dish or multiwell plate) at 4° C. overnight. The plate was then blocked, followed by incubation of the antibody-producing cells in appropriate dilutions (usually between 10³ to 10⁶ cells/ml), for 12 to 16 hours at 37° C. in a humidified incubator (containing 5% CO₂). Detection of the antigen-antibody complex at the site of the active antibody-secreting cell was accomplished by incubating for 2 hours at 37° C. with an enzyme-conjugated, anti-globulin followed by addition of the appropriate substrate (10). The spots were counted at 10× to 30× magnification.

Cytotoxicity Assay for NK Cells

The native lytic activity of NK cells was assessed by lysis of NK-sensitive K562 target cells. Briefly, exponentially growing target cells at 2×10⁵ cells/ml were labeled with 10 μM BrdU (labels the DNA) overnight at 37° C. The labeled target cells (at 1×10⁵ cells/ml) were then mixed with different numbers of effector lymphocytes from the culture in U-bottomed 96-well microtiter plates at 37° C. for 4 hours. Aliquots of the supernatants were collected and BrdU-labeled DNA (released from the lysed target cells) were quantified by sandwich ELISA using the Cellular DNA Fragmentation kit (Boehringer Manheim) as described (11).

Autologous Plasma Collection

Prior to the bone marrow harvest, the volunteers donated 120 ml of peripheral blood that was collected in heparinized tubes to prevent clotting. The peripheral blood was centrifuged at 2000 rpm for 30 min, and the plasma was collected and stored at −20° C. to be used later as needed (10).

Paraffin Thin-Sections

The scaffolding and the cells within were fixed with 10% formaldehyde (Fisher, Pittsburgh, Pa.) for 1 hour at room temperature, embedded in 3% Bacto agar (Gibco), and then immersed in 10% buffered formalin (Fisher). They were then infiltrated with paraffin, thin-sectioned, and stained with hematoxylin/eosin for microscopic examination.

Scanning, Electron Microscopy (SEM)

The scaffolding and the cells within were fixed with 2% formaldehyde and 4% glutaraldehyde mixture in 0.1 M phosphate buffer, washed twice with phosphate buffer, fixed again in 1% OsO₄ water solution for 1 hour, and finally washed with distilled water. The samples were then dehydrated by serial washes with ethanol solution and coated with gold prior to SEM examination (7).

Differential Cell Counts

Slides of the cell samples were prepared using a Cytospin centrifuge (Shandon, Sewickly, Pa.), air-dried prior to staining with Wright's stain. Differential cell counts were performed by counting 100-200 cells in each slide using a light microscope.

Cytochemistry

Formalin-fixed paraffin thin-sections from the culture were labeled with monoclonal antibodies (anti-CD68 for macrophages and anti-CD31 for endothelial cells) or polyclonal antibodies (anti-vimentin for stromal cells of mesenchymal origin), followed by a biotin-conjugated secondary antibody and streptavidin-conjugated peroxidase (DPC). Reticular stromal cells were silver stained and collagen deposition was demonstrated by Masson stain. Endogenous peroxidase activities were quenched by immersing the slides in 3% hydrogen peroxide for 5 minutes prior to the immunostaining (8). Positively stained cells were identified under a light microscope. The morphological characteristics of the positively stained cells were also examined to ensure a consistency with their respective subtypes defined by the cytochemistry.

RNA Arbitrarily Primed PCR (RAP-PCR)

The RNA arbitrarily primed polymerase chain reaction (RAP-PCR) provides a simple and rapid method for fingerprinting RNA gene transcripts. During first-strand synthesis, a single 18-base arbitrary primer (Stratagene, La Jolla, Calif.) anneals and extends from sites contained within the messenger RNA. Second-strand synthesis proceeds in a similar manner during a single round of low-stringency PCR. PCR amplification at high stringency proceeds by virtue of having incorporated the arbitrary primer at both ends of the PCR to amplify the cDNA. A template-dependent competition exists that determines which potential PCR products will ultimately predominate. For every arbitrary primer-RNA combination that is tested, a “mock” first-strand synthesis was conducted in which the reverse transcriptase is eliminated from the reaction. This control sample was subjected to amplification in the subsequent PCR step, thus providing an indication of the background signal derived from the template that did not require reverse transcription.

Analysis of RAP-PCR Products

The resulting RAP-PCR products were analyzed by gel electrophoresis on 6% acrylamide/7 M urea gels (9) which are silver stained using the Pharmacia Silver Stain Kit (Pharmacia, Piscataway, N.J.).

Preparation of the Bioreactor

The bioreactor was fabricated using polycarbonate plates (FIG. 1A). The culture chamber (3/16″H×5/16″W×5/16″L) was packed with 0.01 g of the highly porous microcarriers. The packed-bed of microcarriers was overlayered with culture medium. The medium chamber (½″H×5/16″W×12/16″L) contained 0.6 ml of medium. A Teflon™ membrane (50 μm thickness) was used to facilitate gas exchange.

Cellsnow™-EX, type L (low ion-charged), macroporous cellulose microcarriers (Kirin, Japan; 1-2 mm diameter; 100-200 μm pore size; 95% porosity) were used throughout these experiments as an artificial scaffolding for the human bone marrow cells (FIG. 1B).

Human Bone Marrow Preparation

Bone marrow, aspirated from the iliac crest of consenting donors according to the instructions from the University of Rochester's Research Subjects Review Board, was diluted 1:1 with McCoy's 5A medium (Gibco, Grand Island, N.Y.), overlayered onto Ficol/Paque (Pharmacia, Piscataway, N.J., density 1.027 g/ml), and centrifuged at 200 g for 30 minutes. The mononuclear cell layer was collected, washed 3 times, and used to inoculate the bioreactor. A portion of the cells was set aside to be used in various assays as needed.

Three-Dimensional Human Long-Term Bone Marrow Culture

The cultures were inoculated with the proper number of mononuclear cells (4-6×10⁶ cells per culture chamber) by pipetting into the porous microcarrier section of the bioreactor. The cultures were incubated in a humidified CO₂ incubator (containing 5% CO₂) at 37° C. The LTBMC medium (changed daily), consisted of 70% (v/v) McCoy's 5A medium (Gibco), 1×10⁶ M hydrocortisone (Sigma, St. Louis, Mo.), 50 u/ml penicillin (Sigma), 50 mg/ml streptomycin (Sigma), 0.2 mM L-glutamine (Gibco), 0.045% sodium bicarbonate (Sigma), 1×MEM sodium pyruvate (Gibco), 1×MEM vitamin solution (Gibco), 0.4×MEM amino acid solution (Gibco), 12.5% (v/v) heat inactivated horse serum (Gibco), and 12.5% heat inactivated FBS (Gibco). The culture medium was supplemented with recombinant human Stem Factor (rhSCF 50 ng/ml) and the lymphocyte-specific lymphokines, interleukin 2 (rhIL-2, 1000 U/ml) and interleukin 7 (rh IL-7, 2 ng/ml). The cultures were fed daily with unsupplemented medium and every second day with the supplemented medium. Feeding with the cytokine-supplemented medium was initiated at day 4.

For the hydrocortisone experiments, the cultures were fed daily with the complete culture medium and starting at day 10 with the hydrocortisone-free medium. For the autologous plasma experiment, the culture medium was supplemented with 10% autologous plasma. At week 2, the cultures were depopulated by gently stirring and mixing the bed of porous microspheres to release the non adherent cells (50 μl/well). Viable cell count for the nonadherent cells was determined by the dye-exclusion method using Trypan blue dye (Sigma) and a hemocytometer. The cultures were harvested at week 3, gentle pipetting and sacrificed at week 4 to perform the various assays.

### Detection of B Lymphocytes

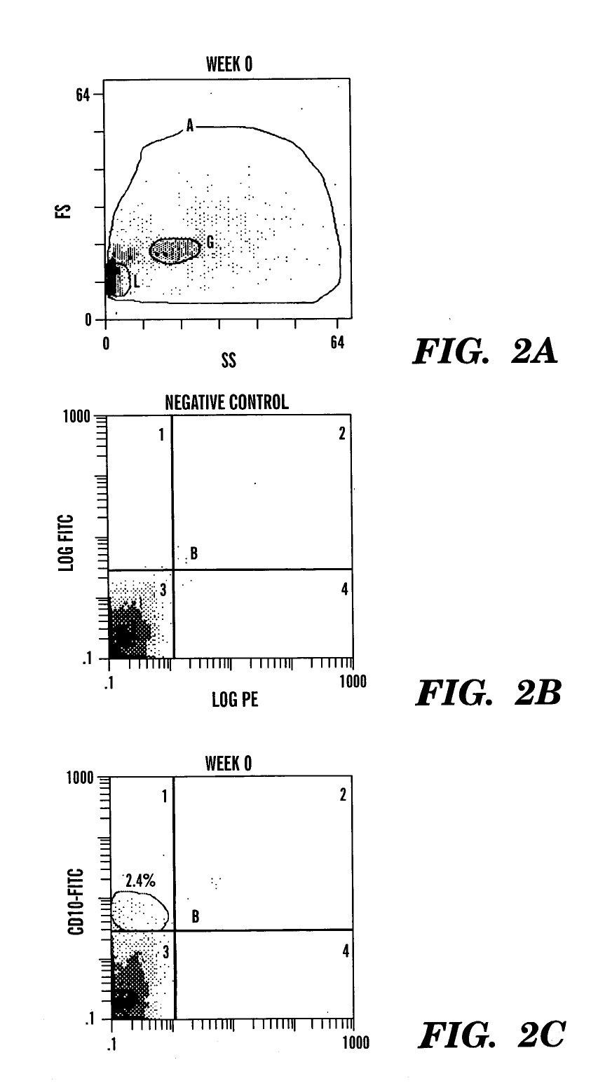

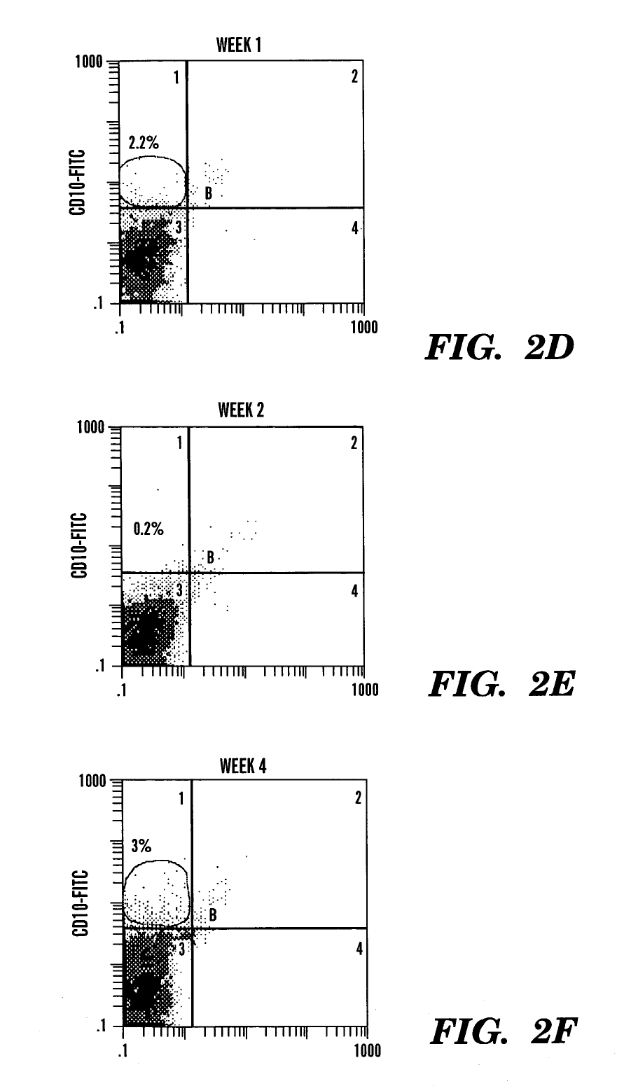

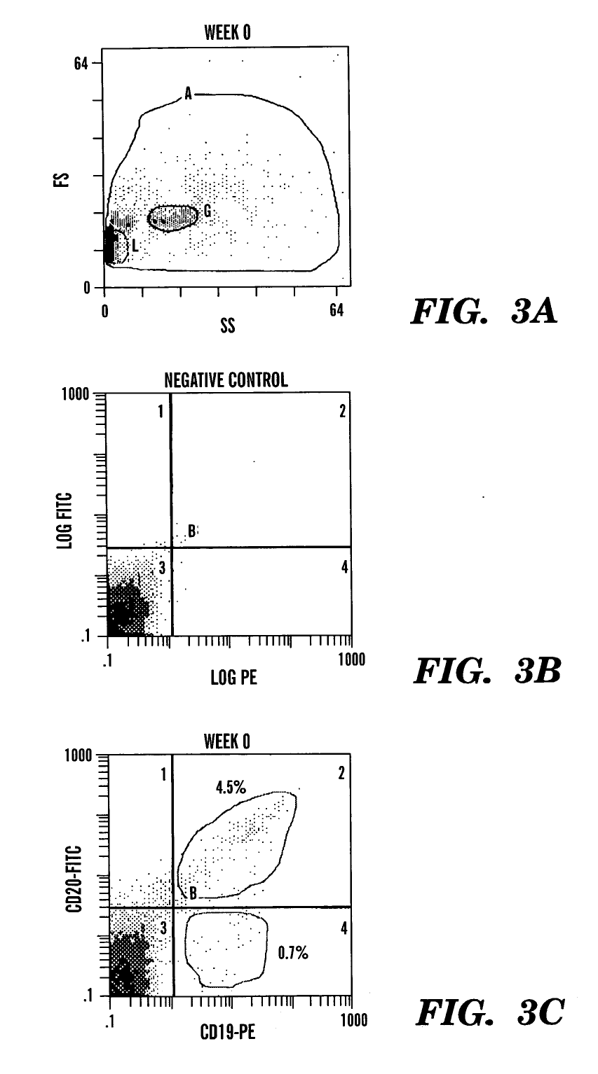

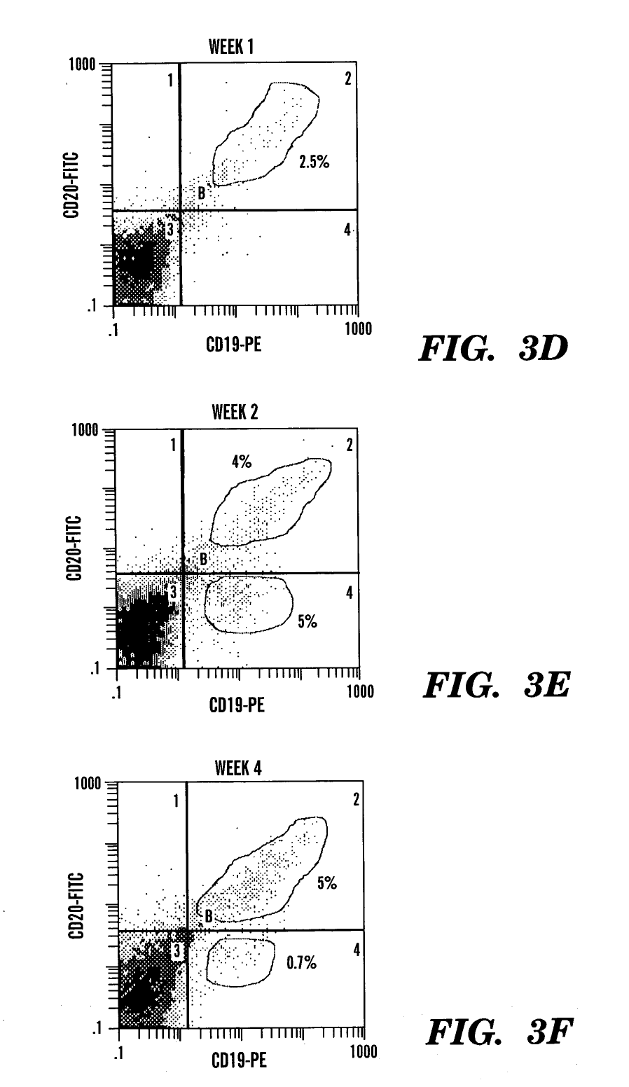

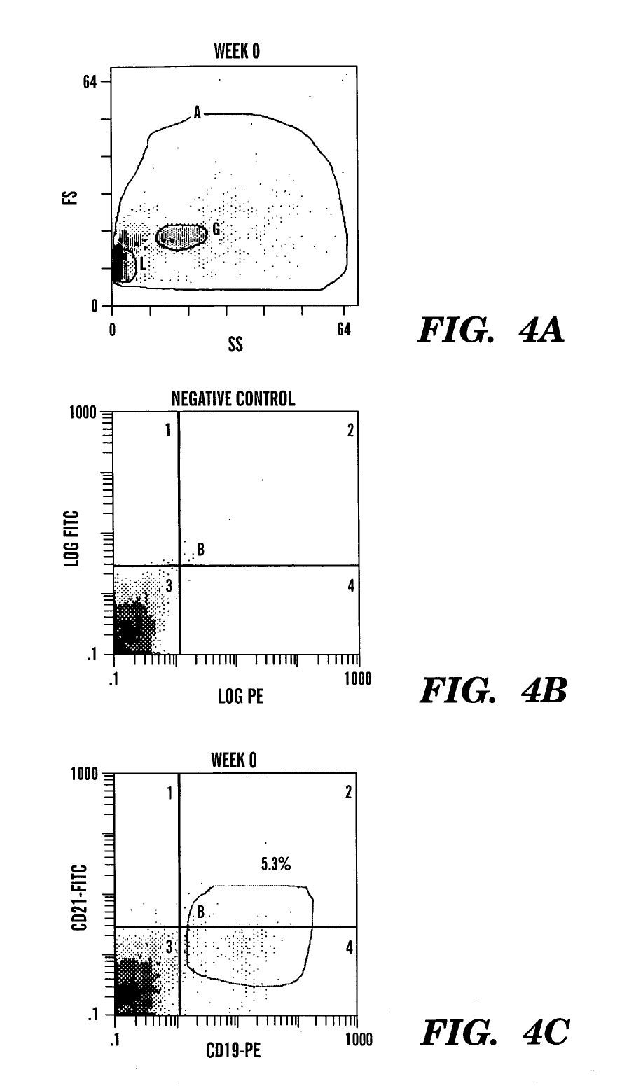

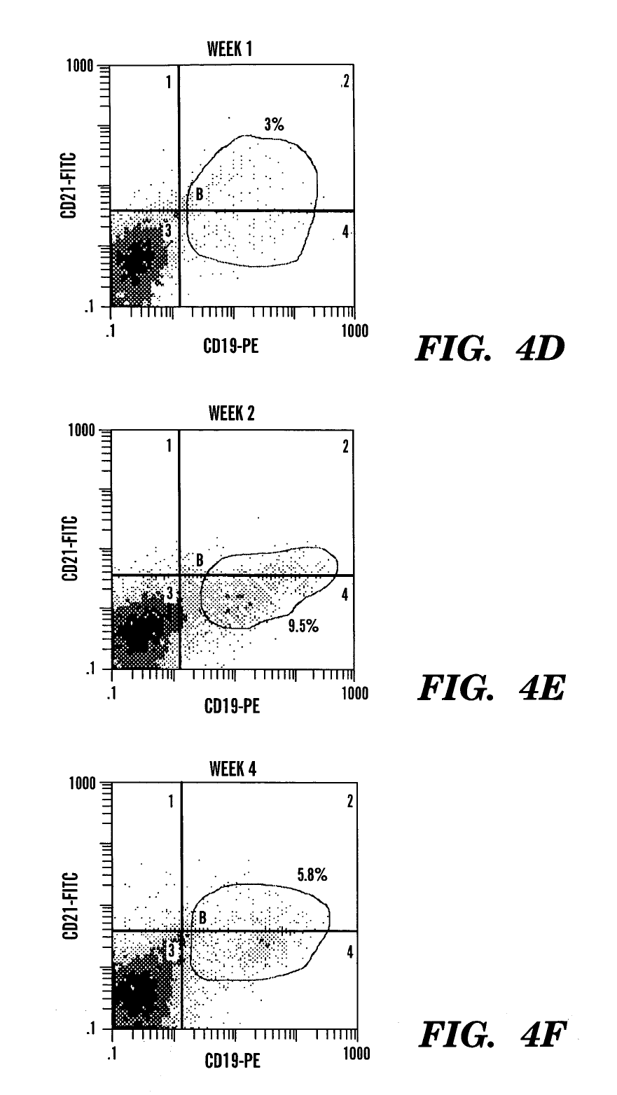

Flow cytometric analysis of the cell-output from the three-dimensional human bone marrow mimicry confirmed the presence of pro-B (CD10⁺), immature B (CD19⁺), and mature B-cells (CD20⁺, CD21⁺), in the absence of exogenous growth factors. At week 0 2.4% of the cells expressed the CD10 marker, representing the pro-B cell population in the fresh marrow (FIG. 2). After one week of culture, the pro-B cell population was maintained at the same levels. However, at week 2 the pro-B cell population decreased dramatically, only to recover by week 4. This fluctuation probably represents a regeneration process that occurs in the three-dimensional culture and signifies the active B cell lymphopoiesis present in the bioreactor. Furthermore, the fluctuation in the CD10⁺ B cell population (pro-B cells) corresponded with fluctuations in the immature (CD19⁺) and mature (CD20⁺ and CD21⁺) B cells. Specifically, at week 0 the CD19⁺ CD20⁺ B cell population was 5.7% (FIG., 3). At week 1, the CD19⁺ CD20⁺ population decreased by half to 2.5%. At week 2, the CD19⁺ CD20⁺ had recovered and expanded to 9%. This recovery corresponded with the decrease in the CD10⁺ cells and most likely represents the maturation of B cells from pro-B cells to immature (CD19⁺) and mature (CD19⁺ CD20⁺) B cells. At week 4, the levels of CD19⁺ and CD20⁺ cells were at the same point as fresh marrow. In a similar fashion, FIG. 4 shows the expression of the CD19 and CD21 B cell markers. Throughout the culture, there was little expression of the CD21 marker (which represents B cells at the last stage of maturation), in a fashion similar to marrow in vivo. It is worth noticing the excellent agreement of the total B cell population using the different B lymphocyte markers.

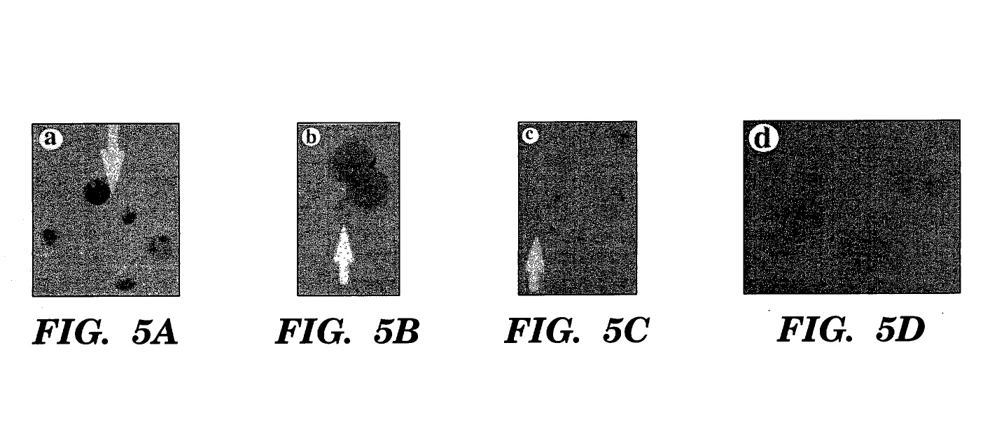

To examine for the presence of lymphoid stem cells and pre-B cells, immunocytochemistry was employed. FIG. 5a confirmed the presence of lymphoid stem cells in the three-dimensional bioreactor (stained positive for nuclear TdT). TdT+ cells represent a small percentage (0.1%) of the cells in the bone marrow. Pre-B lymphocytes were also present throughout the culture period as determined by the cytoplasmic :-positive cells. The functionality of the B-cells produced in the bioreactor was examined using the ELISPOT assay. FIGS. 5c and 5d show that the B-cells in the culture (week 4) were able to secret antibodies upon activation by lipopolysaccharide (LPS), indicating that the B-cells were functional. The human marrow culture therefore appears to support ex vivo B-lymphopoiesis, again resembling the function of marrow in vivo. This provides an exciting and unprecedented opportunity to study the microenvironment for human lymphopoiesis.

### Detection of T Lymphocytes

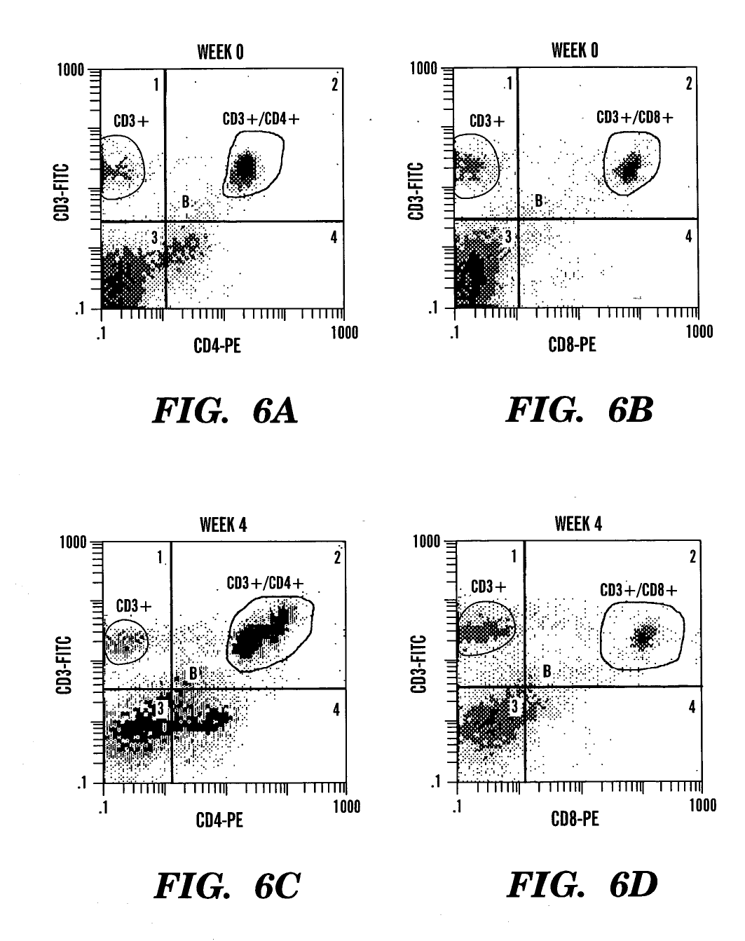

Flow cytometric analysis of the cell-output from the three-dimensional human bone marrow bioreactor indicated that most of the lymphocytes (>90%) identified in the differential count were CD3⁺ T-cells. Further analysis showed that both subtypes of T cells were present (FIG. 6). In particular, helper T-cells (CD3⁺, CD4⁺) and cytotoxic T-cells (CD3⁺ CD8⁺) were present throughout the culture period in the absence of exogenous growth factors. This observation further points out the ability of the human three-dimensional bone marrow mimicry to support lymphopoiesis ex vivo.

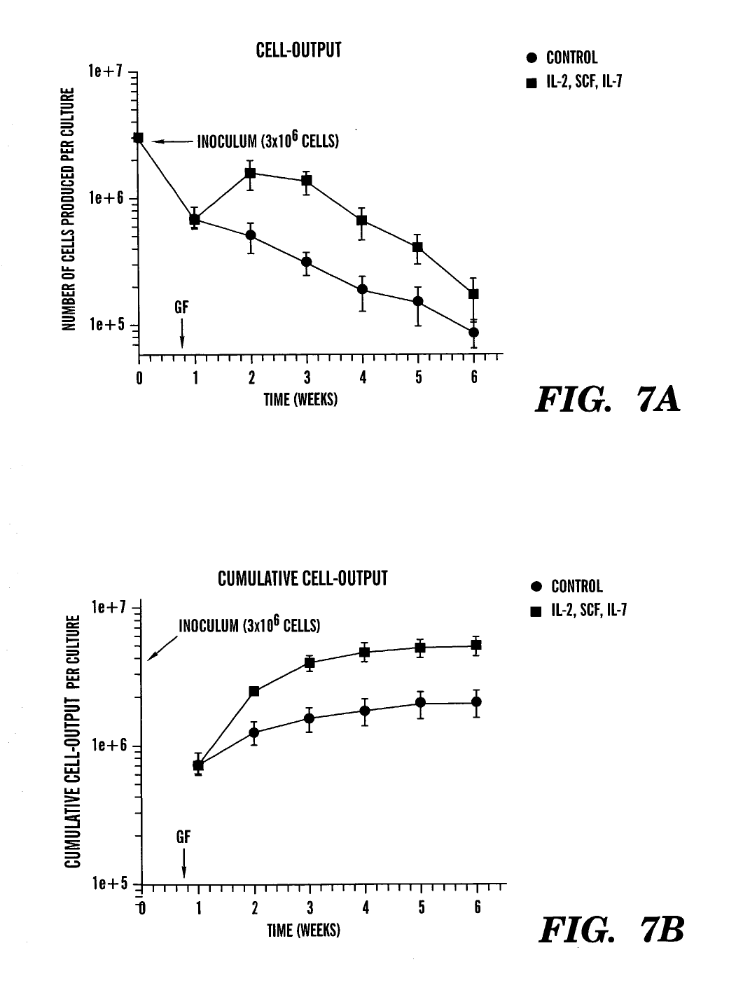

To further characterize the T lymphocytes present in the cell-output from the bioreactor, a series of experiments was performed in the absence and presence of exogenous lymphocyte-specific cytokines (lymphokines). Specifically, interleukin-2 (rh IL-2, 1000 U/ml) and interleukin-7 (rh IL-7, 2 ng/ml) were supplemented in the culture medium, as well as stem cell factor (rh SCF, 50 ng/ml). FIGS. 7a and 7b show that the cell-output from the cultures supplemented with cytokines was stimulated as compared to the control (without cytokines) by a factor of 2-8. Furthermore, in the presence of cytokines, the cumulative cell-output exceeded the inoculum by week 3 suggesting the expansion and/or production of T lymphocytes in the bioreactor. More important, the addition of the lymphokines resulted in a sustained expansion for 5 weeks. Differential cell analysis (Table 1) confirmed that the expansion in the cell-out in the presence of the lymphocyte-specific cytokines was in the lymphoid population. At week 2, the lymphoid cells constituted the majority of the cells (55%). Similarly, at week 4, the lymphocyte population accounted for 58.7% of the cell-output, a 3 fold increase when compared to the control. Therefore, the addition of the lymphokines resulted in a shift in hemopoiesis in the bioreactor towards lymphopoiesis.

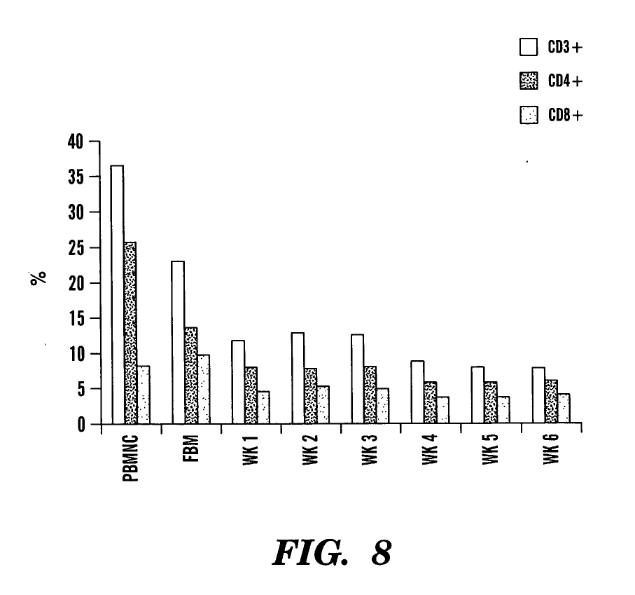

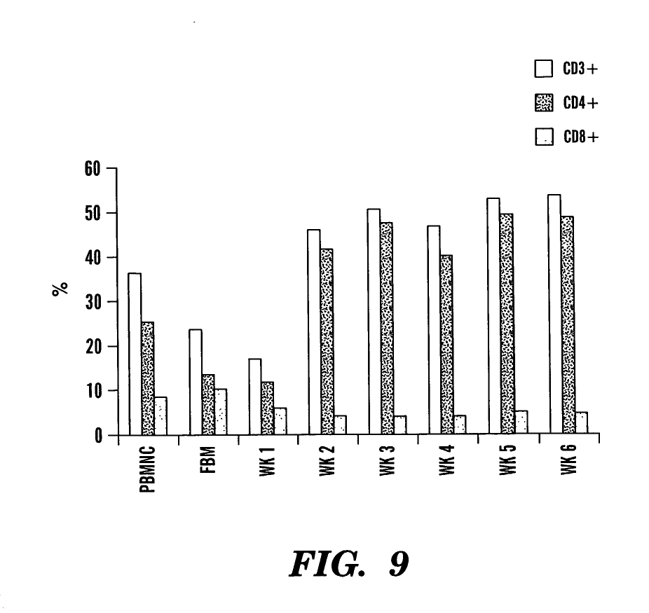

The T-cell subtypes were also analyzed using flow cytometry by following the expression of the CD3, CD4, and CD8 antigens. In the absence of exogenous growth factors (FIG. 8), the percentage of T lymphocytes decreased from 25% in the fresh marrow (week 0) to approximately 10-15% during the culture period. Interestingly, the ratio of CD4⁺ helper T-cells to CD8⁺ cytotoxic T-cells remained constant throughout the culture period at a ratio of 1.5:1, which is the normal ration in the bone marrow in vivo. When exogenous lymphocyte-specific cytokines (IL-2 and IL-7) along with SCF were supplemented in the culture medium, the preferential stimulation of CD4⁺ T-cells was observed (FIG. 9). This can be explained by the fact that IL-2 is known to stimulate helper T-cells which in turn produce cytokines that further enhance the helper T-cell population.

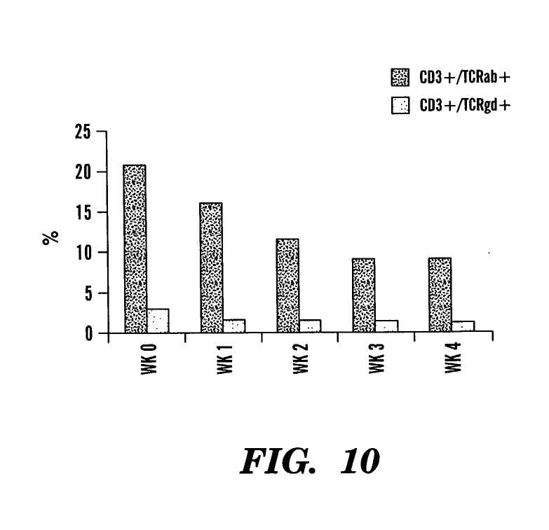

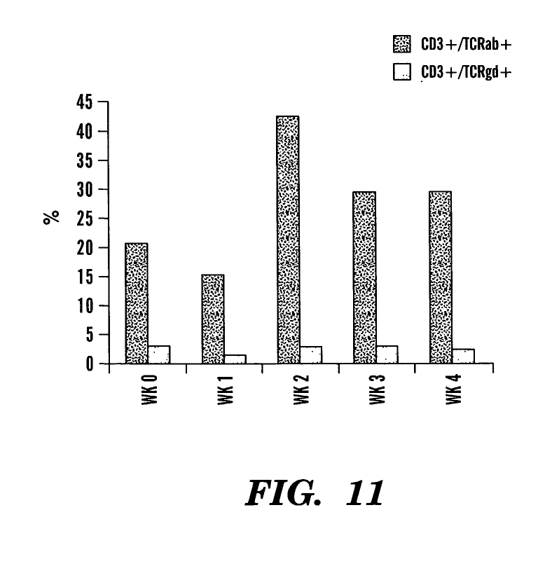

The T-cell receptor (TCR) subtype was also investigated. T-cells are known to have two TCR subtypes expressed on their surface, TCRαβ and TCRγδ. Most T-cells express the αβ TCR. FIG. 10 shows that the majority of T cells (95%) expressed the αβ TCR on their surface in the absence of growth factor. When SCF, IL-2, and IL-7 were added to the culture medium, the T lymphocytes expanded and/or expressed the αβ TCR. In contrast, the T cells expressing the γδ TCR were not stimulated.

The data confirm the presence of T-cells in the cell-output from the human bone marrow model. Both T-lymphocyte subtypes, helper and cytotoxic, were present (in the absence of exogenous growth factors) at a ratio that was similar to the bone marrow in vivo. Moreover, most T-cells expressed, as expected, the αβ TCR. Furthermore, the T cells in the bioreactor were stimulated in a manner consistent with their subtype when exogenous lymphokine-specific growth factors were added, indicating that these cells are functional. Therefore, the three-dimensional human bone marrow model produces a microenvironment that is conducive to lymphopoiesis and offers exciting opportunities for delineating the signals, molecules, and cellular interactions crucial for the development of lymphocytes.

### Effects of Hydrocortisone Removal

In the Whitlock-Witte culture, hydrocortisone, a known immunosuppressant, has to be removed for B-lymphopoiesis. Furthermore, removal of hydrocortisone in flask cultures results in the decline of the cell-output. However, in the three-dimensional human bone marrow model, both Band T-lymphocytes wee present even in the presence of hydrocortisone. In this example, the effects of hydrocortisone removal on ex vivo lymphopoiesis were examined. In doing so, optimal conditions for active lymphopoiesis were determined. In addition, the gene expression patterns of the hydrocortisone-containing and hydrocortisone-free cultures were also compared in order to identify potential genes that are associated with hydrocortisone removal. Hydrocortisone was deleted from the medium at day 0, day 3, or between 10-14.

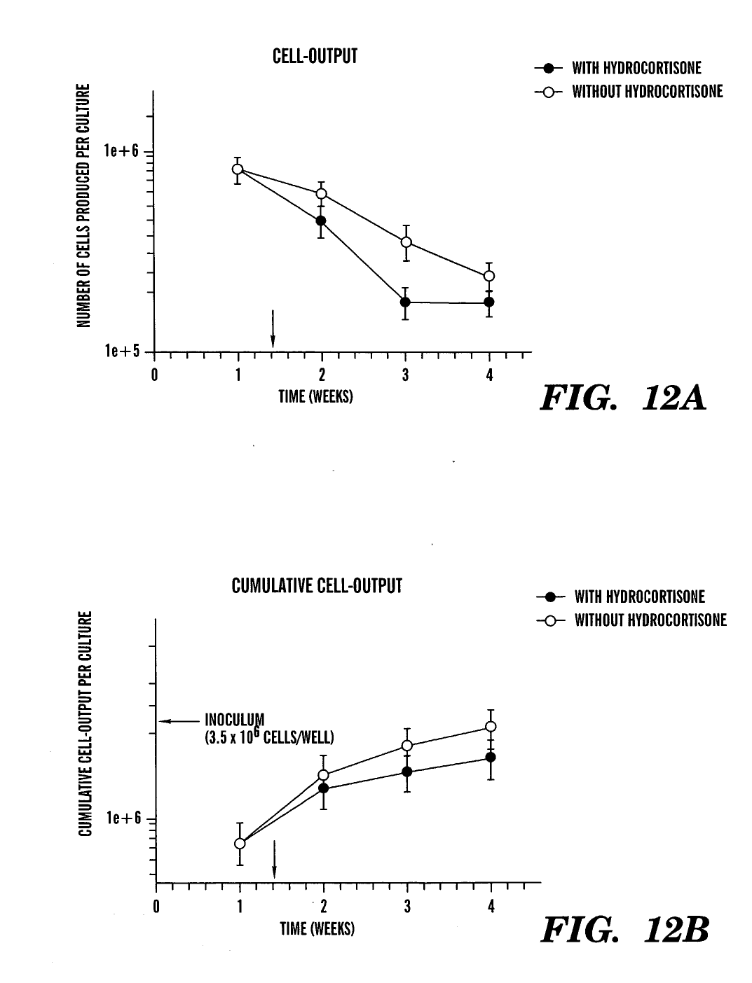

The timing of hydrocortisone removal was crucial for the survival of the cultures. When hydrocortisone was removed at day 0 or day 3, the cultures collapsed soon thereafter. However, removal of hydrocortisone at day 10 or 14 resulted in stable cultures that maintained healthy viabilities (>80%). FIG. 12 shows that removal of hydrocortisone at day 10, resulted in the increase in the cell-output by week 2. The increase in the cell-output was maintained throughout the culture, with week 3 being the most dramatic. The stimulation of the cell-output in the three-dimensional mimicry is in sharp contrast with the traditional flask cultures where the cell-output drops. The viability, after the removal of hydrocortisone, remained high and comparable to the hydrocortisone-containing cultures at 85-95%.

Differential cell analysis was performed to identify any potential effects of hydrocortisone removal on specific cell types. Hydrocortisone withdrawal from cultures from two independent donors (at week 3) appeared to enhance immature lymphocytes and granulocytes (Table 2). Specifically, myeloblasts and myelocytes showed a two-fold increase, whereas lymphoblasts increased by 50%. This result is in agreement with the role of hydrocortisone as an immunosuppressant.

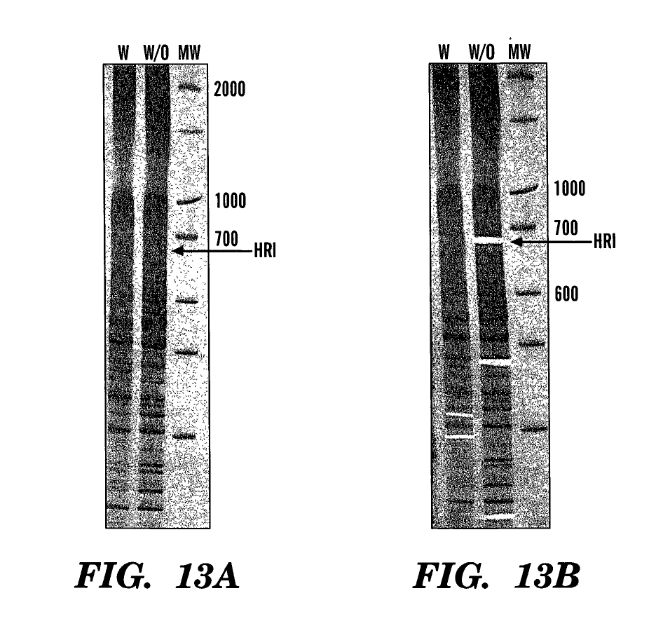

Differential display techniques (RAP-PCR) were used to examine any differences in the gene expression pattern between hydrocortisone-containing and hydrocortisone-free cultures in order to identify any genes related to hydrocortisone removal. Three-dimensional human bone marrow cultures, with and without hydrocortisone, were sacrificed at week 4 and the mRNA from the adherent cells was analyzed for differential displayed genes. FIG. 13 confirmed that withdrawal of hydrocortisone resulted in a different gene expression pattern. Among the genes identified thus far is the heme-regulated initiation fact 2 alpha kinase gene. The gene was identified by excising one of the differentially displayed gene fragments (FIG. 13, 682 bp), re-amplified, cloned, and sequenced. Heme controls the synthesis of protein in reticulocytes. The heme-regulated eukaryotic initiation factor 2 alpha (eIF-2α), also called heme-regulated inhibitor (HRI), plays a major role in this process (59, 60).

These results demonstrate the significance of hydrocortisone as a supplement in the culture medium and its role on hemopoiesis. Moreover, the significance of the three-dimensional bioreactor as a tool for elucidating the role of modulators, such as hydrocortisone, on hemopoiesis was also illustrated.

### Use of Autologous Plasma

The use of autologous plasma as a substitute for animal sera in the culture medium was studied. Animal sera contain foreign proteins that potentially could activate or suppress cell differentiation and proliferation. Autologous plasma circumvents this problem. However, its limited availability presents a challenge for long-term cultures. Therefore, the feasibility of autologous plasma was evaluated by examining two concentrations (5 and 10%) of plasma. Cell-output and cell differentiation was investigated. In addition, B-and T-cells were monitored using flow cytometry by following the expression of CD19 and CD3 antigens.

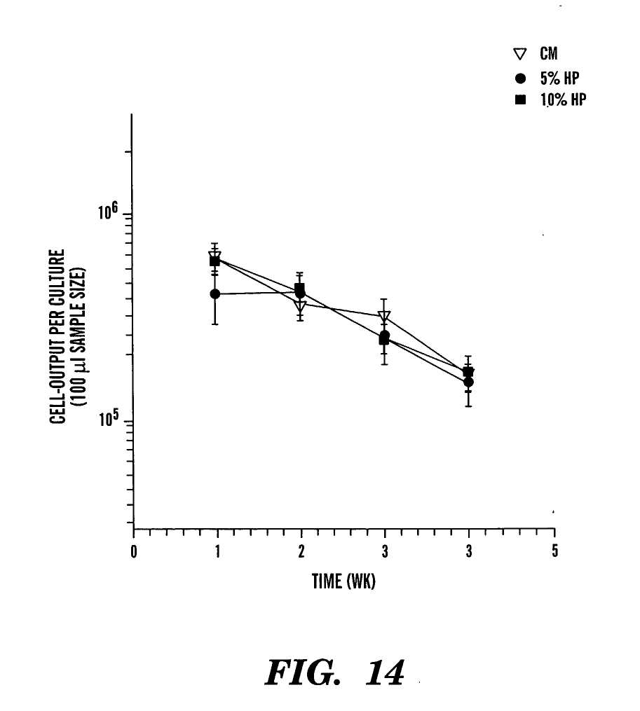

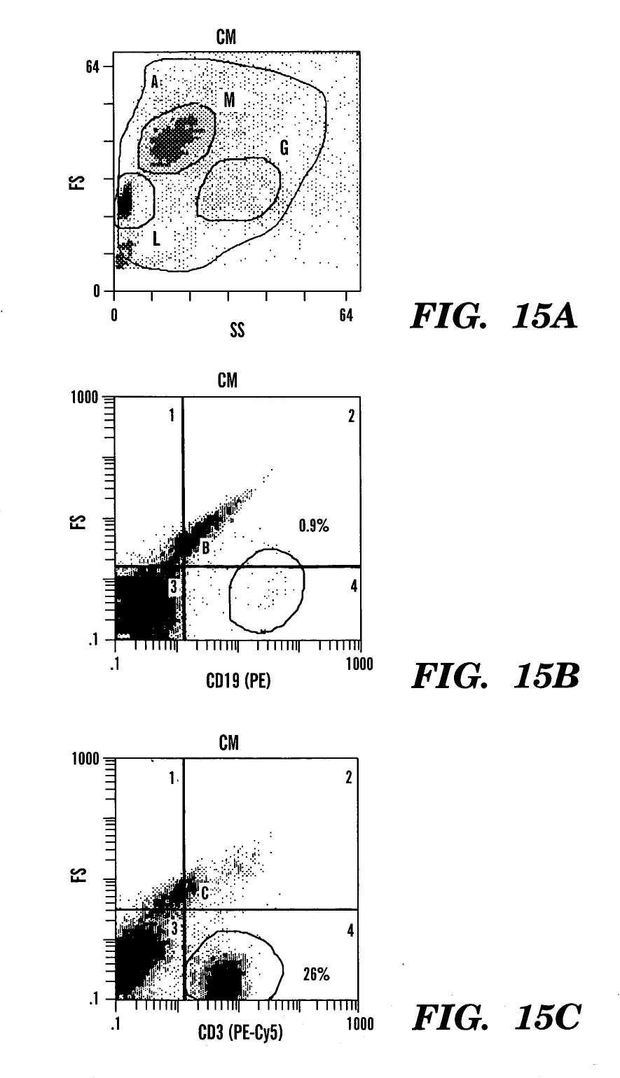

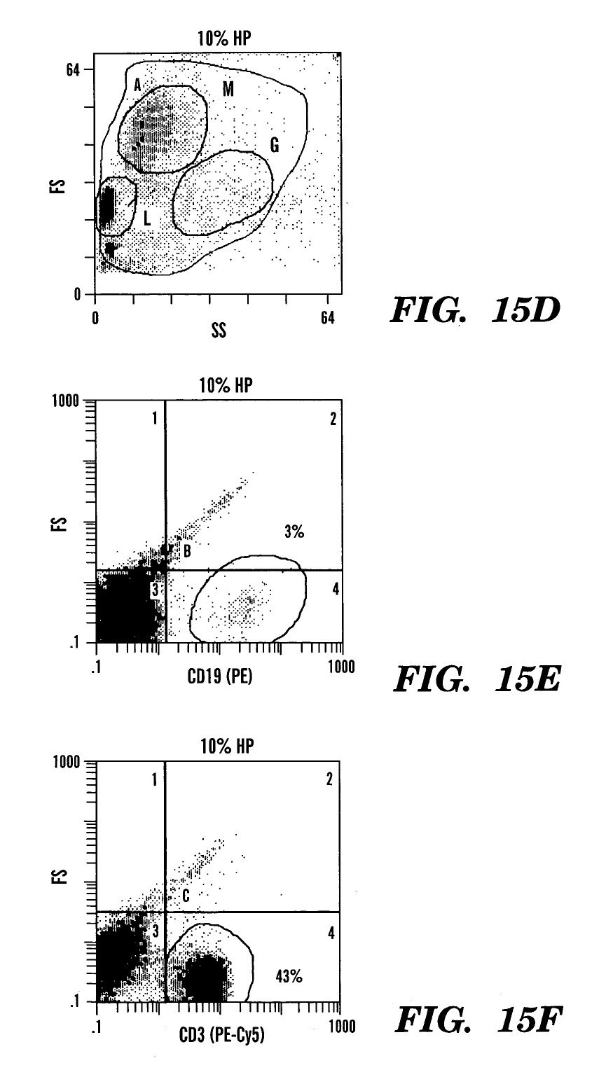

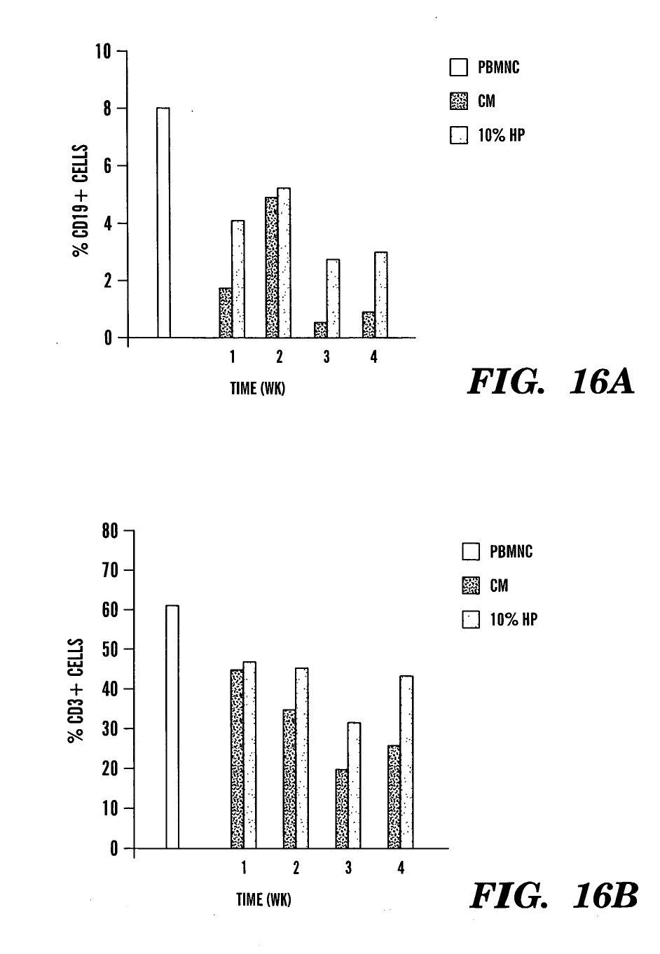

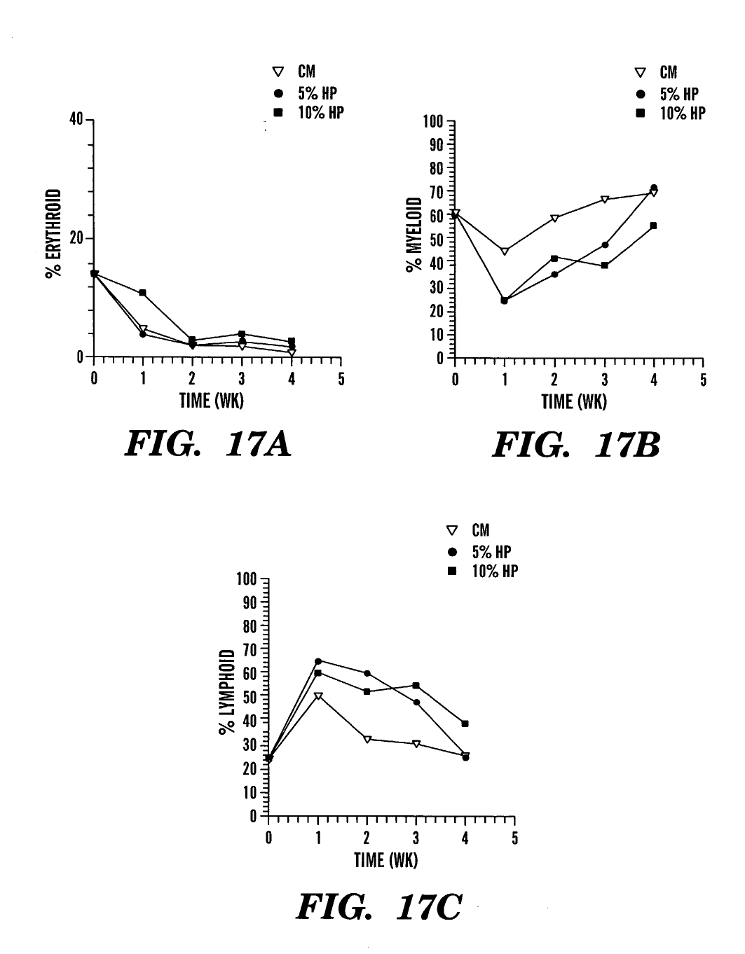

Cell output kinetics from the three-dimensional cultures indicated that the use of autologous plasma (at both 5% and 10%) was not affected when compared to animal sera-supplemented media cultures (FIG. 14). Flow cytometric analysis of the immature B cells (CD19⁺) and T cells (CD3⁺) suggested that autologous plasma supported the lymphocyte populations better than animal sera containing media (FIG. 15). Specifically, more immature B cells were present in the 10% autologous plasma cultures than the control cultures (FIG. 34), especially during weeks 3 and 4. A similar trend was observed with the T cell population (FIG. 16). Finally, differential cell kinetic analysis confirmed the observation that the autologous plasma (especially the 10% containing cultures) enhanced the lymphocyte population (FIGS. 17a-17c).

These data demonstrated the feasibility of using autologous plasma as a substitute for animal sera in long-term human bone marrow cultures. As such, this would allow for the study of hemopoiesis under more physiological conditions void of foreign antigenic stimulation or suppression.

### References

(1) Dexter T. M. and Lajtha. Brit J Haematol 28: 525 1974.

(2) Hocking W. G. and Golde D. W. 1980. Blood. 56: 118.

(3) Slovick F. T., Abboud C. N., Brennan J. K., Litchman M. A. (1984). Survival of granulocytic progenitors in the nonadherent and the adherent compartments of human long-term bone marrow cultures. EXP Hematol 12: 327.

(4) Whitlock, C. A., D. Robertson, and O. N. Witte. 1984. Murine B cell lymphopoiesis in long term culture. J. Immunological Methods. 67: 353-369.

(5) Whitlock C. A., Witte O. N. 1982. Long-Term culture of B lymphocytes and their precursors from murine bone marrow. Proc. Natl. Acad. Sci. USA, 79: 3608.

(6) Fluckinger, A-C, E. Sanz, M. Garcia-Lloret, T. Su, Q-L. Hao, R. Kato, S. Quan, A. de la Hera, G. M. Crooks, O. N. Witte, and D. J. Rawlings. 1998. In Vitro Reconstitution of Human B-Cell Ontogeny: From CD34+ Multipotent Progenitors to IG-Secreting Cells. Blood. 92(12): 4509-4520.

(7) Tharakan, J. P. and P. C. Chau. 1986. A radial flow hollow fiber bioreactor for the large-scale culture of mammalian cells. Biotechnol. Bioeng. 18: 329.

(8) Elias, J. M. 1984. Immunohistopathology: A Practical Approach to Diagnosis. ASCP Press, Chicago. pp. 1-60.

(9) Sambrook, J., E. F. Fritsch, and T. Maniatis. 1989. Northern hybridization. In: Molecular cloning: A laboratory manual. Second (ed.). Cold Spring Harbor Laboratory Press. pp. 7.39-7.52.

(10) Coligan, J. E., A. M. Kruisbeek, D. H. Margulies, E. M. Shevach, and W. Strober. 1994. In Current Protocols in Immunology. R. Coico, (ed). John Wiley & Sons, Inc., New York.

(11) Pollack, S. B. 1993. Production and Differentiation of NK Lineage Cells in Bone Marrow. Nat. Immun. 12: 177.

What is claimed is:

1. A cell culture system, comprising:
a three dimensional support for the culture of stromal and hemopoietic stem cells; and media which will support the growth of, or differentiation of, the stem cells into immune system cells.

5. The culture system according to claim 1, wherein the hemopoietic stem cells are selected from the group consisting of bone marrow stem cells, peripheral blood stem cells, embryonic stem cells, stem cells from umbilical cord, or stem cells from other sources.

6. The culture system according to claim 1, wherein the immune system cells are selected from the group consisting of T lymphocytes, B lymphocytes, antigen presenting cells, natural killer cells, naive cells, activated cells, memory cells, and progenitors or precursors thereof.

7. The culture system according to claim 6, wherein the T lymphocytes comprise at least one of CD4⁺, CD8⁺, CD3⁺, or TdT⁺ cells.

8. The culture system according to claim 6, wherein the T lymphocytes have αβ or γδ T cell receptors.

9. The culture system according to claim 6, wherein the B lymphocytes comprise at least one of CD19⁺, CD20⁺, CD21⁺, CD10⁺, TdT⁺, CD5⁺, Ig⁺, cytoplasmic mu chain⁺ or plasma cells.

10. The culture system according to claim 6, wherein the antigen presenting cells are selected from the group consisting of macrophages and dendritic cells.

11. The culture system according to claim 1, wherein the media contains cytokines, extracellular matrices, or other biologically active molecules.

12. The culture system according to claim 11, wherein the cytokines are selected from the group comprising interleukin-2, interleukin-4, interleukin-6, interleukin-7, interleukin-12, flt-3L, stem cell factor, thrombopoietin, interleukin-4, CD40L, BCA-1, L-BCGF, and soluble interleukin-6R.

13. The culture system according to claim 1, further comprising non-bone marrow cells or cell lines.

14. The culture system according to claim 13, wherein the non-bone marrow cells comprise peripheral blood immune cells.

106. A method of cell growth and expansion which comprises culturing stromal and hemopoictic stem cells on a three dimensional support and allowing for the growth of, or differentiation into, immune system cells; transfecting the immune system cells, and inoculating a further culture with the transfected cells.
